(19) United States
(12) Patent Application Publication
(10) Pub. No.: US 2026/0272390 A1
(43) Pub. Date: Sep. 17, 2026

(54) MULTI-OBJECTIVE ULTRAVIOLET RADIATION EXPOSURE MANAGEMENT SYSTEM

(71) Applicant: UV SENSE, LLC, Salt Lake City, UT (US)

(72) Inventors: Kristina Schiffman, Salt Lake City, UT (US); Kyle Patterson, Salt Lake City, UT (US)

(21) Appl. No.: 19/668,249

(22) Filed: May 5, 2026

Related U.S. Application Data
(63) Continuation-in-part of application No. 18/638,461, filed on Apr. 17, 2024, now Pat. No. 12,624,992.
(60) Provisional application No. 63/496,526, filed on Apr. 17, 2023.

(51) Int. Cl. A61B 5/00; A61B 5/11; G01J 1/42

(52) U.S. Cl. CPC A61B 5/7275; A61B 5/1118; A61B 5/441; A61B 5/6803; A61B 5/6824; A61B 5/746; G01J 1/429; G01J 2001/4266

(57) ABSTRACT

An apparatus, system, and method for dynamically managing UV radiation exposure using multi-objective threshold determination, dual-output dose calculation, and biomarker-calibrated benefit tracking are disclosed. The system includes a wearable UV sensor assembly, a processor, and storage media. A dynamic user model is configured to maintain a continuous fractional photosensitivity score derived from weighted inputs and updateable over time. An effective UV exposure value is processed through a first calculation pathway applying an erythemal action spectrum to produce a burn-prevention output and a second calculation pathway applying a synthesis efficacy curve to produce a Vitamin D synthesis output simultaneously. A multi-objective evaluator module is configured to compare both outputs against exposure-related thresholds including a risk threshold associated with erythemal damage and a synthesis target calibrated based on serum 25-hydroxyvitamin D level. A conflict detector module is configured to identify threshold inconsistencies, and an optimization logic module is configured to resolve conflict. The system integrates clinician-prescribed parameters, escalating notifications, activity-adaptive body mapping, angular correction, and longitudinal synthesis tracking.

### CROSS-REFERENCE TO RELATED APPLICATIONS

This application is a continuation-in-part of U.S. patent application Ser. No. 18/638,461, filed on Apr. 17, 2024, which claims the benefit of U.S. Provisional Application Ser. No. 63/496,526, filed on Apr. 17, 2023. The entire contents of each of the foregoing applications are incorporated herein by reference in their entirety.

### FIELD

The present disclosure relates generally to ultraviolet (UV) radiation exposure management and more particularly relates to systems, methods, and computer-readable media for dynamically managing the UV radiation using multi-objective threshold determination, dual-output dose calculation, and biomarker-calibrated benefit tracking.

### BACKGROUND

Ultraviolet (UV) radiation exposure presents significant health risks, including sunburn, premature skin aging, and increased risk of skin cancer. Existing approaches to monitoring UV exposure often rely on generalized environmental data or standalone devices that provide limited or intermittent feedback. Wearable UV sensing devices have been developed to provide more localized and real-time monitoring, however such devices face challenges related to measurement accuracy under dynamic conditions. Factors such as device orientation, sensor placement, user movement, and environmental variability may adversely affect sensor readings, and existing systems may lack mechanisms for validating or adapting to these conditions. While some systems assess UV exposure risk based on physical characteristics and body mapping data, such systems have generally focused on a single objective: preventing erythemal skin damage. However, UV radiation exposure presents a multi-objective challenge that existing systems do not adequately address.

UV-B radiation, while responsible for the majority of acute erythemal damage and a significant contributor to skin cancer risk, is also the primary driver of cutaneous Vitamin D synthesis. Vitamin D deficiency is prevalent and underdiagnosed, with contributing factors including geographic latitude, indoor lifestyle patterns, widespread sunscreen use, darker skin tones, and advancing age. A fundamental biological tension exists between the UV exposure required for adequate Vitamin D synthesis and the UV exposure that produces erythemal skin damage. Existing UV monitoring systems do not identify this conflict, quantify it, or generate actionable guidance to resolve it.

Furthermore, existing systems typically assign a fixed skin type classification at onboarding and do not dynamically update the user's photosensitivity profile over time to reflect short-term adaptive responses such as tanning or long-term cumulative photodamage. Existing systems also generally operate in isolation from clinical oversight, with no mechanism for clinicians to monitor patient compliance, set prescribed UV dose targets, UV avoidance ceilings, or concern area designations that integrate directly with a patient's UV monitoring system. Additionally, existing systems lack the ability to account for the dynamic degradation of protective substances such as sunscreen, the effect of user activity on body exposure geometry, the angular orientation of a sensor relative to solar incidence, or environmental factors such as air quality and atmospheric attenuation that modify the relationship between measured UV and actual erythemal risk.

Accordingly, there remains a need for an improved UV radiation exposure management system that dynamically manages multiple exposure objectives simultaneously, maintains a dynamic user model reflecting the user's evolving photosensitivity, detects and resolves conflicts between competing exposure-related thresholds, integrates clinical oversight, accounts for dynamic degradation of protective substances and environmental modifiers, and tracks cumulative exposure and synthesis accumulation longitudinally.

### SUMMARY

A program product including a non-transitory computer-readable storage medium and program code for dynamically managing ultraviolet (UV) radiation exposure of a user is disclosed. A system and method may also perform the functions of the program product.

According to one aspect of the present disclosure, the program product includes a non-transitory computer-readable storage medium storing program code executable by one or more processors to perform operations. The operations include receiving input data associated with a user, the input data including radiation-related data derived from a wearable UV sensor assembly, environmental data, user-related data, and/or protection-related data. The operations further include maintaining a dynamic user model associated with the user. The dynamic user model is updateable over time based on the input data, and the dynamic user model represents a current response of the user to UV exposure. The operations further include determining, based on the dynamic user model and the input data, multiple exposure-related thresholds. The exposure-related thresholds include a first threshold associated with a risk condition and a second threshold associated with a benefit condition. The operations further include determining an effective UV exposure value based on the input data and at least one variable modification factor. The operations further include evaluating a multi-objective exposure condition by comparing the effective UV exposure value to the exposure-related thresholds.

The operations further include detecting, in response to at least one of the exposure-related thresholds being inconsistent with at least one other of the exposure-related thresholds, a conflict condition. The operations further include utilizing an optimization logic based on the dynamic user model to resolve the conflict condition. The operations further include generating an output configured to influence user behavior based on the resolved conflict condition.

In some embodiments, the operations further include integrating external radiation data from a remote data source. In some embodiments, the dynamic user model is updated using a machine learning process.

In certain embodiments, the effective UV exposure value include a time-weighted cumulative exposure. The output may include a predictive alert generated prior to reaching the risk threshold. In some embodiments, the operations further include generating a user interface including a visualization of exposure and threshold conditions.

According to another aspect of the present disclosure, the system includes a wearable UV sensor assembly configured to detect UV radiation data associated with a user. The system further includes one or more processors and non-transitory computer-readable storage media storing code. The code is executable by the one or more processors to receive input data associated with the user. The input data includes radiation-related data derived from the wearable UV sensor assembly, environmental data, user-related data, and/or protection-related data. The operations further include maintaining a dynamic user model associated with the user. The dynamic user model is updateable over time based on the input data, and the dynamic user model represents the current response of the user to UV exposure.

The operations further include determining, based on the dynamic user model and the input data, multiple exposure-related thresholds. The exposure-related thresholds include a first threshold associated with the risk condition and a second threshold associated with a benefit condition. The operations further include determining an effective UV exposure value based on the input data and the at least one variable modification factor and evaluating the multi-objective exposure condition by comparing the effective UV exposure value to the exposure-related thresholds.

The operations further include detecting, in response to the at least one of the exposure-related thresholds being inconsistent with at least one other of the exposure-related thresholds, the conflict condition. The operations further include utilizing an optimization logic based on the dynamic user model to resolve the conflict condition. The operations further include generating an output configured to influence user behavior based on the resolved conflict condition. In these and other embodiments, the output may include at least one of a notification, a recommendation, a behavioral instruction, and a control signal.

In some embodiments, the radiation-related data includes ultraviolet A (UV-A) radiation data and/or ultraviolet B (UV-B) radiation data. The UV-A radiation and the UV-B radiation data are independently obtained from separate sensor elements. The variable modification factor may include a user-configurable concern weighting configured to adjust a relative contribution of the UV-A radiation data and the UV-B radiation data to the effective UV exposure value.

In some embodiments, the environmental data includes geographic location, time of day, altitude, atmospheric conditions, and/or weather data. In certain embodiments, the protection-related data includes information associated with a protective substance applied to the user. The information may include a formulation type of the protective substance and a time-dependent degradation of the protective substance modeled as a function of an elapsed time, an accumulated UV dose, a perspiration level, and/or an activity intensity.

In some embodiments, the dynamic user model includes a representation of a skin type, pigmentation level, and/or a physiological response to UV exposure. The dynamic user model may be continuously updated based on historical exposure data associated with the user.

In some embodiments, the first threshold corresponds to a risk of skin damage and the second threshold corresponds to a target associated with Vitamin D synthesis. In some embodiments, the output includes a notification, a recommendation, a behavioral instruction, and/or a control signal.

According to yet another aspect of the present disclosure, a method for dynamically managing the UV radiation exposure of the user includes receiving input data associated with a user. The input data includes at least one of radiation-related data derived from the wearable UV sensor assembly, environmental data, user-related data, and protection-related data. The method includes maintaining a dynamic user model associated with the user. The dynamic user model may be updateable over time based on the input data and may represent the current response of the user to the UV exposure.

The method further includes determining, based on the dynamic user model and the input data, the plurality of exposure-related thresholds, multiple exposure-related thresholds. The exposure-related thresholds include a first threshold associated with a risk condition and a second threshold associated with a benefit condition. The method further includes determining an effective UV exposure value based on the input data and at least one variable modification factor. The method further includes evaluating the multi-objective exposure condition by comparing the effective UV exposure value to the exposure-related thresholds.

The method further includes detecting, in response to at least one of the exposure-related thresholds being inconsistent with at least one other of the exposure-related thresholds, a conflict condition. The method further includes utilizing an optimization logic based on the dynamic user model to resolve the conflict condition. The method further includes generating an output configured to influence user behavior based on the resolved conflict condition.

In some embodiments, determining the effective UV exposure value includes applying an orientation-based adjustment on the radiation-related data to generate a corrected UV reading used in determining the effective UV exposure value. The orientation-based adjustment may be derived from a three-dimensional (3D) orientation of the wearable UV sensor assembly and a calculated sun position. The orientation-based adjustment may be activity-aware based on an expected device orientation profile associated with a detected activity type.

In some embodiments, the method further includes estimating a biologically effective UV dose based on a spectral component of the effective UV exposure value. In some embodiments, the method further includes estimating a Vitamin D synthesis level based on the biologically effective UV dose, the synthesis efficacy curve, and an exposed body surface area. The exposed body surface area may be derived from an activity profile and/or a body diagram interface input.

In some embodiments, the method further includes generating a report aggregating UV exposure data across sessions and body areas. The report may include behavioral compliance analysis, predictive modeling of future exposure risk, and/or a projected Vitamin D synthesis accumulation at a current behavioral trajectory.

In some embodiments, resolving the conflict condition includes prioritizing one of the exposure-related thresholds based on user-specific parameters, and generating a fractionated exposure recommendation distributing a target UV dose across multiple shorter sessions. Each session may remain within the risk threshold, such that cumulative synthesis across the plurality of shorter sessions meets a synthesis target. In some embodiments, the output includes a staged notification based on proximity to one or more thresholds.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a schematic plan diagram illustrating one embodiment of a UV radiation exposure notification system including a wearable UV sensor assembly and a computing device in accordance with some embodiments of the disclosure;

FIG. 2 is an exploded perspective view of one embodiment of wearable UV sensor assembly including a processor, a UV sensor, a power supply, and a communication device contained within a housing assembly in accordance with certain embodiments of the disclosure;

FIG. 3 is a perspective view of the wearable UV sensor assembly of FIG. 2 assembled in accordance with some embodiments of the disclosure;

FIG. 4 is a perspective view of one embodiment of the wearable UV sensor assembly that includes a wristband configured to be worn by a user in accordance with some embodiments of the disclosure;

FIG. 5A is a perspective view of another embodiment of the wearable UV sensor assembly that includes an armband configured to be worn by a user in accordance with some embodiments of the disclosure;

FIG. 5B is a perspective view of the wearable UV sensor assembly of FIG. 5A rotated to another position on the user's arm in accordance with some embodiments of the disclosure;

FIG. 6 is a front view of another embodiment of the wearable UV sensor assembly that includes an attachment element configured to be attached to an item in accordance with some embodiments of the disclosure;

FIG. 7 is a perspective view of one embodiment of the wearable UV sensor assembly that includes an attachment element configured to be attached to the top of a helmet in accordance with some embodiments of the disclosure;

FIG. 8 is a schematic plan diagram illustrating body mapping data for positioning a wearable UV sensor assembly to correspond to any of various areas of a user's body in accordance with some embodiments of the disclosure;

FIG. 9 is a front view of a representative computing device including one embodiment of an apparatus configured to display a query regarding a physical characteristic of a user and various selectable input options in response to the query in accordance with some embodiments of the disclosure;

FIG. 10 is a front view of a representative computing device including one embodiment of a display element configured to display a remaining time for safe UV radiation exposure in accordance with some embodiments of the disclosure;

FIG. 11 is a front view of a representative computing device including one embodiment of an apparatus configured to receive an image of the user as input in accordance with certain embodiments of the disclosure;

FIG. 12 is a plot graph illustrating a time for safe UV radiation exposure relative to a current UV index for use in accordance with some embodiments of the disclosure;

FIG. 13 is a schematic block diagram of a UV radiation exposure monitoring apparatus including various modules in accordance with some embodiments of the disclosure;

FIG. 14 is a schematic block diagram of another embodiment of a UV radiation exposure monitoring apparatus including various modules in accordance with certain embodiments of the disclosure;

FIG. 15 is a flow chart diagram of one embodiment of a method for monitoring UV radiation exposure and notifying a user of a risk of overexposure in accordance with some embodiments of the disclosure;

FIG. 16 is a flow chart diagram of another embodiment of a method for monitoring UV radiation exposure and notifying a user of a risk of overexposure in accordance with some embodiments of the disclosure;

FIG. 17 is a schematic block diagram of a system for dynamically managing UV radiation exposure with simultaneous burn prevention and Vitamin D synthesis tracking capabilities in accordance with some embodiments of the disclosure;

FIG. 18 is a schematic block diagram of the wearable UV sensor assembly configured for dual-band UV radiation detection and orientation sensing in accordance with some embodiments of the disclosure;

FIG. 19 is a schematic block diagram of a dual-output calculation architecture for simultaneous burn prevention and Vitamin D synthesis calculation in accordance with some embodiments of the disclosure;

FIG. 20 is a schematic block diagram of a dynamic user model illustrating weighted inputs for deriving a continuous fractional photosensitivity score and update triggers for modifying the score over time in accordance with some embodiments of the disclosure;

FIG. 21 is a flowchart of a method for multi-objective threshold determination and conflict resolution between exposure-related thresholds in accordance with some embodiments of the disclosure;

FIG. 22 is a schematic block diagram of a Sun Protection Factor (SPF) decay model illustrating inputs and a time-dependent degradation factor for modeling protective substance effectiveness in accordance with some embodiments of the disclosure;

FIG. 23 is a schematic block diagram of an activity-adaptive body mapping system for adjusting per-area UV exposure thresholds based on detected activity type in accordance with some embodiments of the disclosure;

FIG. 24 is a schematic block diagram of an angular correction system for producing corrected UV readings based on device orientation relative to solar incidence in accordance with some embodiments of the disclosure;

FIG. 25 is a schematic block diagram of a clinician portal system for enabling clinical oversight and prescriptive UV dosing capabilities in accordance with some embodiments of the disclosure;

FIG. 26 is a schematic block diagram of an escalating notification architecture illustrating progressive notification stages as a user approaches exposure-related thresholds in accordance with some embodiments of the disclosure;

FIG. 27 is a schematic block diagram of a serum-calibrated Vitamin D tracking system for calibrating synthesis targets based on measured biomarker values in accordance with some embodiments of the disclosure;

FIG. 28 is a flowchart of a fractionated exposure method for resolving conflict conditions by distributing UV dose across multiple shorter sessions in accordance with some embodiments of the disclosure;

FIG. 29 is a schematic block diagram of a multi-profile management system for managing a plurality of linked user profiles including pediatric profiles with age-adjusted thresholds in accordance with some embodiments of the disclosure; and

FIG. 30 is a schematic block diagram of a report generation system for generating customized UV exposure reports including behavioral compliance analysis, predictive modeling, and Vitamin D synthesis projection in accordance with some embodiments of the disclosure.

### DETAILED DESCRIPTION

As discussed above, existing approaches to monitoring ultraviolet (UV) radiation exposure may rely on generalized environmental data or limited sensing capabilities and may not adequately account for variations in exposure across different areas of a user's body, changes in user activity or posture, or dynamic environmental conditions. In addition, factors such as device orientation, sensor placement variability, and user movement can affect the accuracy and reliability of measured UV radiation data, while existing systems may lack mechanisms for validating or adapting to such conditions. Conventional systems may also be limited in their ability to support diverse deployment configurations or broader monitoring contexts beyond individual users. The present disclosure addresses these and other issues.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments.

These features and advantages of the embodiments will become more fully apparent from the following description and appended claims or may be learned by the practice of embodiments as set forth hereinafter. As will be appreciated by one skilled in the art, aspects of the present invention may be embodied as a system, method, and/or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present invention may take the form of a computer program product embodied in one or more computer readable medium(s) having program code embodied thereon.

Many of the functional units described in this specification have been labeled as modules, in order to more particularly emphasize their implementation independence. For example, a module may be implemented as a hardware circuit comprising custom very large scale integrated (“VLSI”) circuits or gate arrays, off-the-shelf semiconductors such as logic chips, transistors, or other discrete components. A module may also be implemented in programmable hardware devices such as a field programmable gate array (“FPGA”), programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations including over different storage devices, and may exist, at least partially, merely as electronic signals on a system or network. Where a module or portions of a module are implemented in software, the program code may be stored and/or propagated on in one or more computer readable medium(s).

The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (“RAM”), a read-only memory (“ROM”), an erasable programmable read-only memory (“EPROM” or Flash memory), a static random access memory (“SRAM”), a portable compact disc read-only memory (“CD-ROM”), a digital versatile disk (“DVD”), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (“ISA”) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (“LAN”) or a wide area network (“WAN”), or the connection may be made to an external computer (for example, through the Internet using an Internet Service Provider). In some embodiments, electronic circuitry including, for example, programmable logic circuitry, field-programmable gate arrays (“FPGA”), or programmable logic arrays (“PLA”) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program instructions may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

The schematic flowchart diagrams and/or schematic block diagrams in the Figures illustrate the architecture, functionality, and operation of possible implementations of apparatuses, systems, methods and computer program products according to various embodiments of the present invention. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

As used herein, a list with a conjunction of “and/or” includes any single item in the list or a combination of items in the list. For example, a list of A, B and/or C includes only A, only B, only C, a combination of A and B, a combination of B and C, a combination of A and C or a combination of A, B and C. As used herein, a list using the terminology “one or more of” includes any single item in the list or a combination of items in the list. For example, one or more of A, B and C includes only A, only B, only C, a combination of A and B, a combination of B and C, a combination of A and C or a combination of A, B and C. As used herein, a list using the terminology “one of” includes one and only one of any single item in the list. For example, “one of A, B and C” includes only A, only B or only C and excludes combinations of A, B and C. As used herein, “a member selected from the group consisting of A, B, and C,” includes one and only one of A, B, or C, and excludes combinations of A, B, and C. As used herein, “a member selected from the group consisting of A, B, and C and combinations thereof” includes only A, only B, only C, a combination of A and B, a combination of B and C, a combination of A and C or a combination of A, B and C.

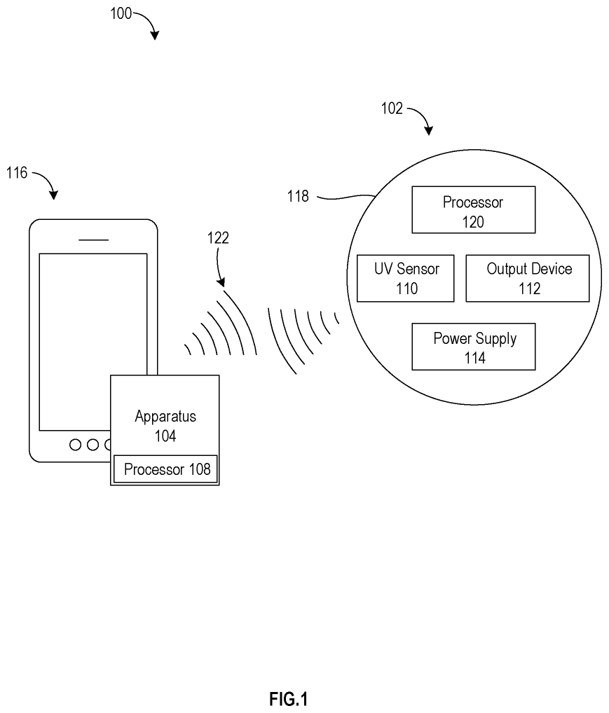

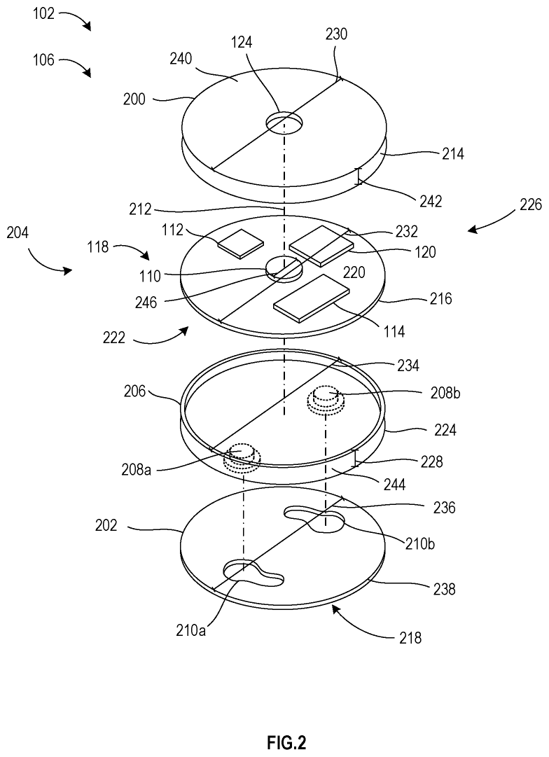

Referring now to FIGS. 1 and 2, a system 100 for monitoring UV radiation exposure and notifying a user of a risk of overexposure is illustrated. In some embodiments, the system 100 includes a wearable ultraviolet (UV) sensor assembly 102 in electronic communication with a computing device 116. The computing device 116 includes a UV radiation exposure monitoring apparatus 104 configured to assess a user's UV radiation exposure and to notify the user of potential overexposure based on the assessment. The wearable UV sensor assembly 102 and the computing device 116 may work together to assist a user in understanding their exposure to UV radiation and may facilitate prevention of skin damage due to overexposure.

In some embodiments, as discussed in more detail below, the wearable UV sensor assembly 102 is configured to receive input and to selectively transmit information, such as a current UV radiation level and exposure time, to a computing device 116. The computing device 116 may include a UV radiation exposure monitoring apparatus 104 and a processor 108. In some embodiments, the computing device 116 is a remote computing device 116 configured to communicate with the wearable UV sensor assembly 102 via a wireless connection 102.

In some embodiments, the wearable UV sensor assembly 102 is further configured to store and/or process at least a portion of the information. In some embodiments, the wearable UV sensor assembly 102 is configured to selectively transmit at least a portion of the information, such as the current UV radiation level and the exposure time, to the processor 108. In some embodiments, the wearable UV sensor assembly 102 is configured to transmit the information to the processor 108 continuously, intermittently, periodically, upon request from the processor 108, and/or at any other desired time. In some examples, selectively transmitting the current UV radiation level includes transmitting only UV radiation measurements that fall within a predetermined range of wavelengths and/or wavelengths within a predetermined range of intensities.

In some embodiments, the wearable UV sensor assembly 102 is configured to operate in temperatures ranging between about −30 to about 80° C., thereby facilitating user participation in a wide variety of outdoor activities including, for example, hiking and biking in desert-like climates and participating in cold-weather sports like skiing or snowmobiling. In some embodiments, the wearable UV sensor assembly 102 is water-resistant and/or incorporates materials configured to protect the device from water damage, thereby facilitating use of the wearable UV sensor assembly 102 in connection with activities in inclement weather such as rain or snow, and/or activities that involve water, such as kayaking or paddleboarding.

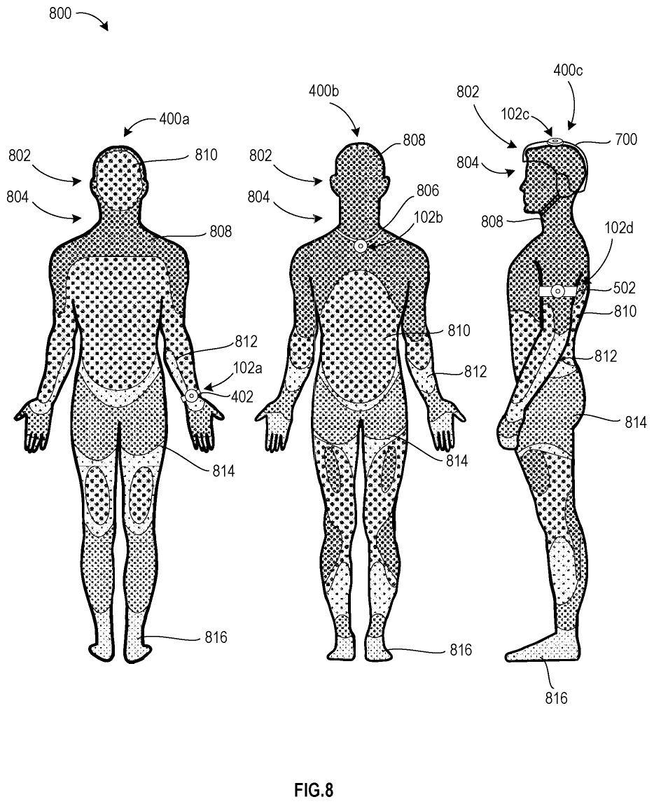

In some embodiments, as shown in FIG. 8, while still referring to FIGS. 1 and 2, the wearable UV sensor assembly 102 is configured to detect a current UV radiation level corresponding to a specified region or area 804 of the user's body. In some embodiments, the wearable UV sensor assembly 102 is adaptable, movable, and/or modifiable such that the wearable UV sensor assembly 102 may be strategically placed in or attached to any of multiple regions or areas 804 of the user's body to detect a UV radiation level corresponding to that area 804.

In certain embodiments, the wearable UV sensor assembly 102 is configured to detect and/or record a UV exposure time corresponding to a region or area of the user's body at which the wearable UV sensor assembly 102 is placed or attached. In other words, in some embodiments, the wearable UV sensor assembly 102 is configured to be worn by the user such that the wearable UV sensor assembly 102 corresponds to at least one of multiple areas of the user's body, and the wearable UV sensor assembly 102 is configured to detect and/or record the amount of time that the area has been exposed to UV radiation.

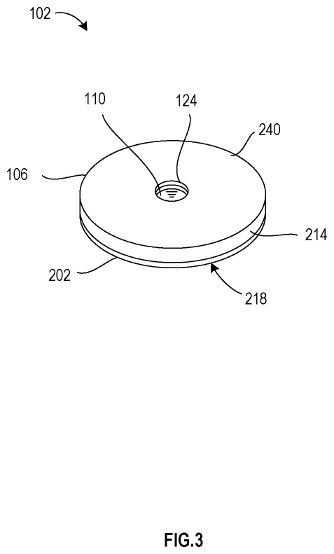

Referring now to FIGS. 2 and 3, while also referring to FIG. 1, in some embodiments, the wearable UV sensor assembly 102 includes one or more component parts 204 including, for example, a printed circuit board 118, a housing lid 200, a housing base 202, and a housing case 206. In some embodiments, the housing base 202 is omitted. In some embodiments, each of the component parts 204 is aligned along a central axis 212. The printed circuit board 118 may be substantially planar and may include an upper surface 220 and a lower surface 222. In some embodiments, the printed circuit board 118 includes a round or substantially circular shape. In other embodiments, the printed circuit board 118 includes any suitable size and/or shape.

In one embodiment, the printed circuit board 118 is a round circuit board having a diameter of about fifty (50 mm) millimeters or less. In certain embodiments, the printed circuit board 118 includes a thickness between about one and about two millimeters (1-2 mm). In one embodiment, the printed circuit board 118 includes a thickness of 1.6 millimeters (1.6 mm). In some embodiments, the printed circuit board 118 includes a double-layer printed circuit board including about one ounce (1 oz) or less of finishing copper. Of course, the printed circuit board 118 may include any suitable materials and/or dimensions. In some embodiments, the printed circuit board 118 may include one or more materials selected to withstand a specific climate or condition, such as warm and/or wet environments.

In some embodiments, the printed circuit board 118 is coupled to various hardware elements 226 via through-hole mounting, surface mount technology, ball grid array, pin grid array, flip-chip technology, wire bonding, a press fit, an adhesive, and/or the like. In these and other embodiments, one or more of the hardware elements 226 may be coupled to the upper surface 220 and/or the lower surface 222 of the printed circuit board 118. In some embodiments, one or more of the hardware elements 226 extends through the printed circuit board 118 between the upper surface 220 and the lower surface 222.

In some embodiments, the hardware elements 226 include a processor 120, a communication device 112, and/or a power supply 114. In some embodiments, the processor 120 includes a microcontroller, a signal processor a field-programmable gate array (FPGA), or the like. In one embodiment, the processor 120 includes an embedded processing system such as Raspberry Pi.

In some embodiments, the wearable UV sensor assembly 102 includes a communication device 112 such as a Bluetooth® chip or other suitable wireless communication device configured to communicate with the processor 108 of the computing device 116. In some embodiments, the communication device 112 is configured to communicate with the computing device 116 via any wireless or near-field communication method known to those in the art, such as Wi-Fi, radio frequency identification (RFID), infrared (IR), Bluetooth®, and/or the like. In some embodiments, the computing device 116 may include a mobile computing device such as a mobile phone, a laptop computing device, a tablet computing device, or the like. In these and other embodiments, a user may carry the computing device 116 on or near his person to facilitate communication with the wearable UV sensor assembly 102 via the communication device 112.

In some embodiments, the power supply 114 includes a lithium-ion battery or other suitable low-energy power supply 114. In one embodiment, the power supply 114 includes a 3.6 V lithium-ion coin cell battery.

In some embodiments, the UV light sensor 110 includes a round UV sensor 110 having a diameter 246 less than the diameter 232 of the printed circuit board 118. The UV light sensor 110 may be attached to the printed circuit board 118 by any suitable fastening device or technique. In some embodiments, the UV light sensor 110 is substantially centered with respect to the printed circuit board 118 and/or the central axis 212. In these and other embodiments, the UV light sensor 110 may be exposed through an aperture 124 disposed through the housing lid 200. In some embodiments, the UV light sensor 110 and the aperture 124 include substantially congruent shapes. In other embodiments, the UV light sensor 110 includes dimensions at least slightly less than the aperture 124 to maximize exposure of the UV light sensor 110 to UV radiation through the aperture 124.

In some embodiments, the UV light sensor 110 is configured to identify UV light within the UV-A and the UV-B spectrums. In certain embodiments the UV light sensor 110 is configured to identify UV light having a wavelength between about two hundred eighty nanometers (280 nm) and about four hundred nanometers (400 nm). In some examples, the UV light sensor 110 includes a narrowband UV sensor configured to detect UV radiation within a specific wavelength range. In some examples, the UV light sensor 110 is configured to differentiate between UV-A and UV-B radiation. In some examples, the UV light sensor 110 includes at least one of a photodiode UV sensor, a photomultiplier tube (PMT) UV sensor, and/or any combination thereof. In some examples, the UV light sensor 110 is a broadband UV sensor.

In some embodiments, the printed circuit board 118 and its various hardware elements 226 are encased within a housing assembly 106. The housing assembly 106 may include any suitable size and/or shape and may be assembled such that each of the component parts 204 aligns along a central axis 212. In some embodiments, one or more of the component parts 204 includes complementary features configured to selectively engage one another to hold the housing assembly 106 together and/or to provide structural support to the housing assembly 106. In some embodiments, for example, one or more of the component parts 204 includes threads, grooves, and/or pronged elements configured to fit, twist, and/or otherwise lock into place when engaged with a corresponding feature of another component part 204. In one embodiment, at least one component parts 204 of the housing assembly 106 includes a three-pronged part configured to be inserted into a corresponding space in another component part 204 to provide twist-lock capabilities.

In certain embodiments, one or more attachment elements 208a, 208b may be configured to extend through more than of the component parts 204 to hold the housing assembly 106 together and/or to provide structural support. In some embodiments, an attachment element 208a, 208b includes, for example, a pole, a screw, a bolt, a rivet, a grommet, a clip, an adhesive, and/or any other suitable mechanical feature or device known to those in the art. In certain embodiments, the housing assembly 106 is monolithically formed as a single unit.

In some embodiments, the housing assembly 106 includes a housing case 206, a housing lid 200, and/or a housing base 202. In certain embodiments, the housing case 206 is disposed between the housing lid 200 and the housing base 202. In some embodiments, the housing assembly 106 includes a substantially rigid or semi-rigid material configured to withstand UV radiation and mechanical stresses. In some embodiments, for example, the housing assembly 106 includes a suitable thermoplastic material such as acrylonitrile butadiene styrene (ABS) and/or polycarbonate (PC). In other embodiments, the housing assembly 106 includes one or more suitable synthetic materials such as polyethylene (PE), polypropylene (PP), acrylic (PMMA), polyethylene terephthalate (PET), polyoxymethylene (POM), and/or the like.

In some embodiments, the housing case 206 includes a shape and/or dimensions such that the housing case 206 substantially surrounds the printed circuit board 118 and its associated hardware elements 226. In some embodiments, the housing case 206 is coupled to or integrated with the housing base 202. In some embodiments, the housing base 202 includes a size and shape substantially identical to the size and shape of the printed circuit board 118 such that the printed circuit board 118 is disposed directly on top of and aligned with the housing base 202 within the housing assembly 106. In some embodiments, one or more attachment elements 208a, 208b is configured to couple one or more of the component parts 204 together when the wearable UV sensor assembly 102 is assembled.

In some embodiments, the housing case 206 is defined by a wall 224 extending substantially parallel to the central axis 212. The housing case 206 may be coupled to the housing base 202 such that the wall 224 extends substantially perpendicularly from an outside edge 238 of the housing base 202. In one embodiment, the wall 224 defines a circular shape having a diameter 234 at least slightly greater than a diameter 232 of the printed circuit board 118. In this manner, the wall 224 may be disposed adjacent to and may substantially circumscribe a periphery 216 of the printed circuit board 118. In some embodiments, a height 228 of the wall 224 defines a depth of the housing assembly 106.

In some embodiments, the housing case 206, the housing lid 200 and the housing case 206 include substantially congruent shapes. In some embodiments, the housing lid 200 includes a top surface diameter 230 at least slightly greater than the diameters 232, 234, 236 of the printed circuit board 118, the housing case 206, and housing base 202. In some embodiments, the housing lid 200 includes a lip 214 extending substantially perpendicularly from the top surface 240 of the housing lid 200. In some embodiments, the lip 214 is configured to extend at least partially over and adjacent to an outer surface 244 of the wall 224 of the housing case 206 to encase the printed circuit board 118 within the housing assembly 106. In some embodiments, the lip 214 includes a height 242 substantially identical to or greater than a height 228 of the wall 224.

In some embodiments, the housing lid 200 includes a cutout or aperture 124 substantially corresponding the size, shape, and/or position of the UV light sensor 110 on the printed circuit board 118. In these and other embodiments, at least a portion of the UV light sensor 110 may be directly exposed to UV radiation through the housing lid 200 when the housing assembly 106 is fully assembled.

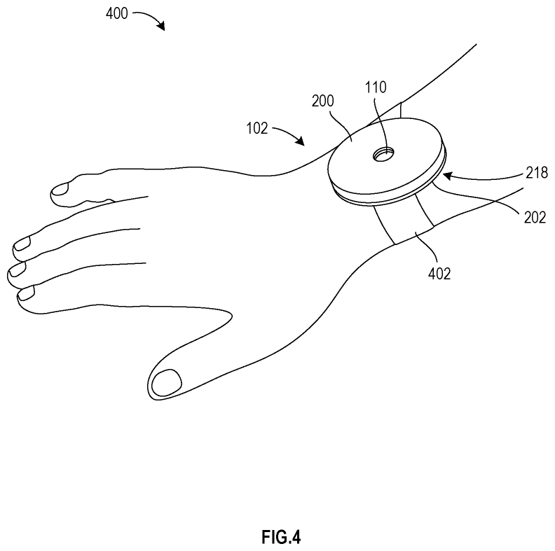

Referring now to FIGS. 4, 5A, 5B, 6, and 7, in some embodiments, the housing base 202 and/or another component parts 204 or portions of the housing assembly 106 includes one or more features or attachment points configured to secure or couple an accessory or attachment device to the wearable UV sensor assembly 102. In some embodiments, for example, an accessory or attachment device includes an armband, a wristband, a headband, a loop, a clip, a helmet, a necklace, a lanyard, a combination thereof, and/or the like. In some embodiments, as shown in FIG. 2, the housing base 202 and/or other component part 204 includes one or more apertures 210a, 210b configured to receive at least a portion of strap of the accessory or attachment device therethrough.

As illustrated in FIG. 4, while also referring to FIG. 2, in some embodiments, the wearable UV sensor assembly 102 is attached to a user 400 via a wristband 402. In some embodiments, a length of the wristband 402 extends through the apertures 210a, 210b to integrate the wearable UV sensor assembly 102 with the wristband 402. In some embodiments, the wristband 402 may include a substantially flexible, wear-resistant material such as silicone, nylon, polyester, leather, rubber, vinyl, fabric, and/or any other suitable material. In some embodiments, the wristband 402 includes an elastic material. In certain embodiments, the wristband 402 is substantially planar and includes dimensions configured to comfortably fit various users.

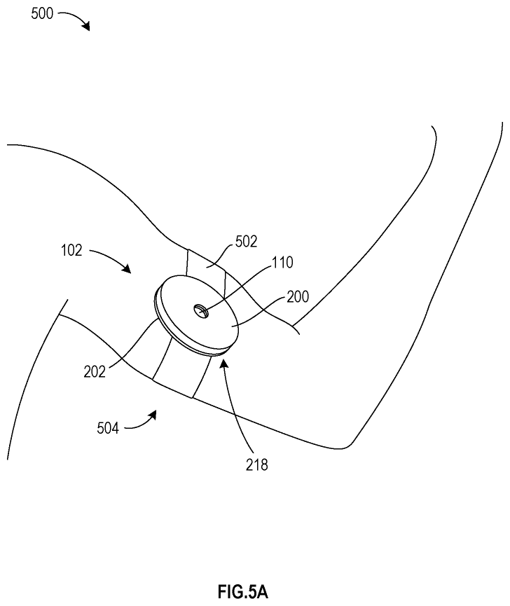

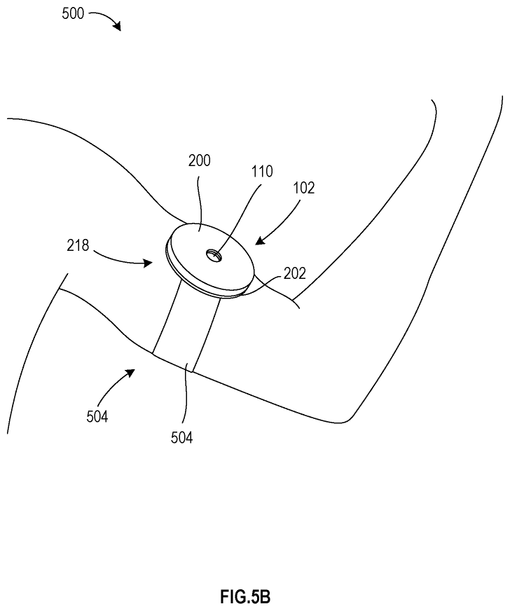

Similarly, in some embodiments as shown in FIGS. 5A and 5B, while also referring to FIG. 2, the wearable UV sensor assembly 102 is attached to a user 500 via an armband 502. In some embodiments, a length of the armband 502 extends through the apertures 210a, 210b to integrate the wearable UV sensor assembly 102 with the armband 502. In some embodiments, the armband 502 may include a substantially flexible, wear-resistant material such as silicone, nylon, polyester, leather, rubber, vinyl, fabric, and/or any other suitable material. In some embodiments, the armband 502 includes an elastic material and/or may be adjusted via a suitable mechanical feature or device such as a hook and loop fabric or the like. In some embodiments, the wearable UV sensor assembly 102 is rotatable with respect to the armband 502. In these and other embodiments, the wearable UV sensor assembly 102 may be selectively rotated to ensure that the UV light sensor 110 is exposed to direct sunlight and/or to maintain comfort. In certain embodiments, the armband 502 is substantially planar and includes dimensions configured to comfortably fit various users.

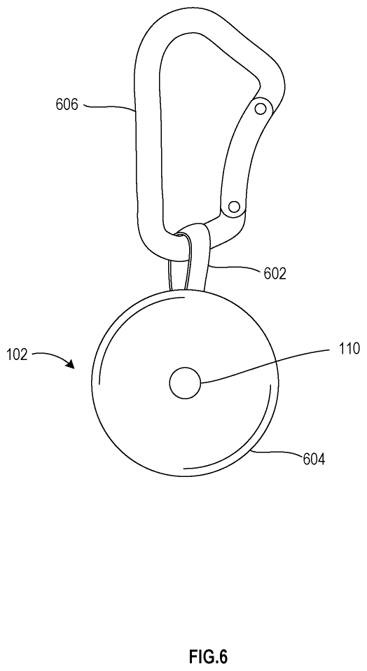

Referring now to FIG. 6, in some embodiments, the wearable UV sensor assembly 102 is configure to attach to a carabiner or other suitable clip or attachment device 606. In these and other embodiments, the wearable UV sensor assembly 102 may be attached to a variety of secure locations on the body, garments, or accessories. In some embodiments, the housing base 202 or other component part 204 of the housing assembly 106 is securely coupled to a looped attachment point 602 via one or more suitable mechanical fasteners such as a bolt, a rivet, a grommet, an adhesive, and/or the like. In some embodiments, at least a portion of the looped attachment point 602 is configured to extend laterally beyond a perimeter 604 of the wearable UV sensor assembly 102. In some embodiments, the attachment device 606 is configured to be coupled to the wearable UV sensor assembly 102 by threading the attachment device 606 through the looped attachment point 602. In this manner, the attachment device 606 may couple the wearable UV sensor assembly 102 to any of various garments, items, or accessories as desired. In other embodiments, any suitable accessory may be threaded through the looped attachment point 602 to facilitate attaching the wearable UV sensor assembly 102 to a desired item. For example, the accessory may include a necklace, a lanyard, a strap, or any other suitable item.

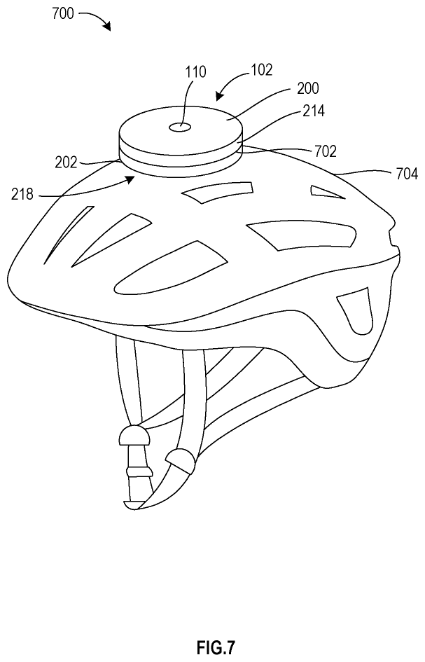

Referring now to FIG. 7, in some embodiments, the wearable UV sensor assembly 102 is mounted to a helmet 700 via a rigid mounting element 702 coupled to or integrated with a portion of the helmet 700. In one embodiment, the mounting element 702 is mounted to a top surface 704 of the helmet 700. In some embodiments, the mounting element 702 is removably or fixably coupled to the housing base 202 or other portion of the housing assembly 106 to mount the wearable UV sensor assembly 102 to the helmet 700. In some embodiments, the mounting element 702 includes a curvature substantially corresponding to a curvature of the top surface 704 of the helmet 700. In this manner, the wearable UV sensor assembly 102 may be securely maintained against the top surface 704 of the helmet 700 for secure and reliable use during various activities including, for example skiing, biking, snowmobiling, and the like.

Referring now to FIG. 8, while also referring to FIG. 1, in some embodiments, the wearable UV sensor assembly 102 is versatile and may be used in any of multiple regions or areas 804 of a user's 400a, 400b, 400c body 802. In some embodiments, wearable UV sensor assembly 102 is configured to be attached to various items or accessories to facilitate positioning the wearable UV sensor assembly 102 to correspond to a desired area 804. As discussed above, such items or accessories may include a wristband 402, an armband 502, a helmet 700, a necklace, a lanyard, a strap, a headband, a leg band, an ankle band, or any other suitable item or accessory.

In some embodiments, the user 400a, 400b, 400c selectively wears, places, or otherwise positions a wearable UV sensor assembly 102 to correspond to an area 804 that is known to be particularly susceptible or prone to UV radiation exposure. In some embodiments, body mapping data 800 may be utilized to facilitate selection of such an area 804.

In some embodiments, body mapping data 800 for the user 400a, 400b, 400c is generated by the processor 108 of the computing device 116. In some embodiments, the body mapping data 800 is based on generalized data as well as on input from the user 400a, 400b, 400c. In some embodiments, the body mapping data 800 is used to assess a UV exposure risk corresponding to each of the areas 804. In some embodiments, the body mapping data 800 divides the body 802 into various areas 804 and indicates the relative susceptibility of each of the areas 804 to skin cancer and/or other damage from UV exposure. In some embodiments, the body mapping data 800 is customized to a user 400a, 400b, 400c based at least partially on input received from the user 400a, 400b, 400c.

As shown, in some embodiments, the body mapping data 800 may include a visual representation of relative susceptibility of various areas 804 of the user 400a, 400b, 400c to skin cancer and/or other damage caused by UV radiation exposure. For example, a first region 808 may have the highest susceptibility to damage from UV radiation exposure, while other areas 804 may indicate decreased relative susceptibility from a second region 810 to a third region 812 to a fourth region 814 and finally to a fifth region 816.

In one embodiment, the user 400a utilizes a wristband 402 to position the wearable UV sensor assembly 102a within the second region 810. In another embodiment, the user 400b utilizes a necklace 806 to position the wearable UV sensor assembly 102b within the first region 808. In another embodiment, the user 400c places more than one wearable UV sensor assembly 102c, 102d to correspond to more than one area 804 within the first region 808. For example, as shown, the user 400c may utilize an armband 502 to position a first wearable UV sensor assembly 102 in one area 804 of the first region 808 and may utilize a helmet 700 to position a second wearable UV sensor assembly 102 in another area 804 within the first region 808.

In some examples, the monitoring apparatus 104 is configured to scan the user 400's body and to present the user 400 with body mapping data 800 customized to the user's 400 body 802. In some examples, the body mapping data 800 is generated without scanning or taking a photo of the user 400 and is instead a generalized representation of the human body 802. In some examples, the monitoring apparatus 104 is configured to receive input from the user 400 regarding particular areas 804 of concern on their body 802. For example, the user 400 can select certain areas 804 of concern via the computing device 116. In some examples, in response to determining that the user 400 has selected an area 804 of concern, the monitoring apparatus 104 is configured to prompt the user 400 400 to upload images of that area 804.

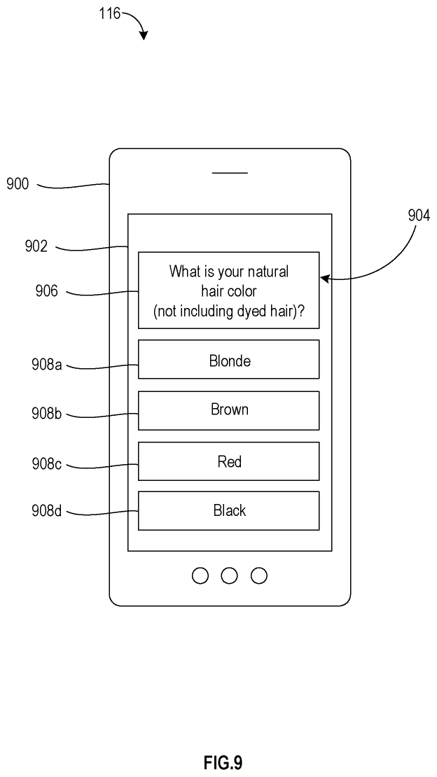

Referring now to FIG. 9, while also referring to FIG. 1, the system 100 includes a computing device 116, such as a laptop computing device, a mobile phone, a tablet computing device, or other suitable computing device 116, in communication with the wearable UV sensor assembly 102 via a wired or wireless connection 122. In some embodiments, the computing device 116 includes a UV radiation exposure monitoring apparatus 104 and a user input device 902. The UV radiation exposure monitoring apparatus 104 may include one or more processors 108 and non-transitory computer-readable storage media storing code executable by the processors 108 to perform operations. The UV radiation exposure monitoring apparatus 104 may be configured to receive user 400 input 908a-908d via the user input device 902. The user input device 902 may include, for example, a touchscreen, a keyboard, a stylus, and/or the like.

In some embodiments, the UV radiation exposure monitoring apparatus 104 is configured to enable the user 400 to initialize a Wi-Fi or other wireless connection 122 between the computing device 116 and a local wireless network. In some embodiments, the UV radiation exposure monitoring apparatus 104 running on the computing device 116 allows the user 400 to initialize and/or customize the wearable UV sensor assembly 102 according to their preferences. The wireless connection 122 may also allow the wearable UV sensor assembly 102 to stream data to a remote server (not shown) either via the computing device 116 or directly through the wireless connection 122.

In some embodiments, the UV radiation exposure monitoring apparatus 104 is configured to receive physical characteristic input 908a-908d regarding at least one physical characteristic of the user 400. In some embodiments, the UV radiation exposure monitoring apparatus 104 generates a questionnaire 904 that includes one or more queries 902 regarding at least one physical characteristic of the user 400. The UV radiation exposure monitoring apparatus 104 may be further configured to display the questionnaire 904 to the user 400 via a display element 900. In some embodiments, the display element 900 includes a display element 900 of a computing device 116, such as a phone, tablet, computer, etc. In some examples, a user interface (e.g., a graphical user interface) is displayed on the display element 900.

In some embodiments, the questionnaire 904 includes one or more queries 906 regarding the user's 400 genetic conditions and/or sun exposure habits. In some examples, the queries 906 are regarding the user 400's natural hair color, eye color, skin color, skin sensitivity, and/or the like. In some examples, the queries include questions related to the user's predisposition to conditions such as erythemal reddening, sun damage, and/or skin cancer. In some examples, a quantity of the queries 906 is not less than five (5) and not greater than twenty (20).

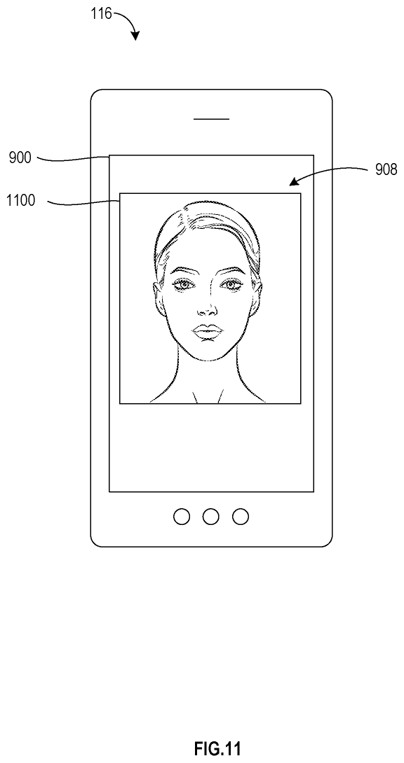

In some embodiments, the UV radiation exposure monitoring apparatus 104 generates a plurality of selectable answers that the user 400 may select as input 908a -908d in response to the query 906. In other embodiments, as shown in FIG. 11, the UV radiation exposure monitoring apparatus 104 is configured to receive from the user 400 one or more images 1100 of various physical characteristics as input 908a-908d.

In some embodiments, the questionnaire 904 includes one or more queries 906 regarding markers related to genetic conditions and/or the user's 400 predisposition to erythemal reddening, sun damage, skin cancer, and/or the like. In some embodiments, such queries 906 facilitates a full understanding of the user's 400 skin, genetics, and/or likelihood of developing sun-related damage. In some embodiments, the genetic queries 906 are used to capture a base condition of the user 400 prior to their use of sunscreen, protective wear, and/or the like. In some embodiments, input 908a-908d provided in response to these queries 906 are weighted more heavily that input 908a-908d provided in response to other queries 906 due to the fact that genetic factors are incredibly important in determining the user's 400 skin type.

In some embodiments, each query 906 will include a unique weight, which may be determined on a sliding scale. For example, if the user 400 is asked “What is the color of your skin?”, the query 906 may include an increased weight when determining the user's 400 skin type than a query 906 regarding the natural color of the user's 400 hair since there is general understanding that, while natural hair color is an important factor in skin type, overall, skin color is more determinative of skin type.

In some embodiments, input 908a-908d provided in response to each query 906 is also weighted. For example, if the user 400 is asked “What is the color of your eyes?”, input 908a-908d indicating the answer choice of “Light Blue” may be weighted more heavily than input 908a-908d indicating “Dark Brown or Black”, since users 400 with darker complexion and features are less likely to have severe reactions to UV radiation exposure. In these and other embodiments, if an answer option indicates the user 400 is more likely to suffer damage from minimal UV exposure, such input 908a-908d may be given greater weight than an answer option indicating that the user 400 is less susceptible to UV damage.

In some embodiments, at least a portion of the questionnaire 904 is directed to habits of the user 400 to further understand how the user 400, given their skin type, operates on a day-to-day basis. Some of these queries 906 may relate to sunscreen use, level of SPF typically used, frequency of protective-wear use, activities the user participates in, and/or the like. In some embodiments, the questionnaire 904 further includes queries 906 relating to occupation, medical treatment, and/or the like.

In some embodiments, the questionnaire 904 is presented to the user 400 intermittently or periodically over the life of the wearable UV sensor assembly 102 and the input 908a-908d is updated accordingly.

In some embodiments, queries 906 are presented in multiple tiers. For example, if a user 400 is asked “How frequently do you wear sunscreen while participating in an outdoor activity?” and the user 400 selects “Always” or “Frequently”, a follow-up question may be asked regarding a level of SPF typically used. In some embodiments, these tiered queries 906 enable the UV radiation exposure monitoring apparatus 104 to first understand the user's 400 habits, and, second, understand the integrity or extent of that habit, to ensure maximum accuracy in calculating their UV exposure threshold.

In one embodiment, a tiered query 906 may regard skin conditions, procedures such as a facial or chemical peel, and/or medications that impact photosensitivity. In this manner, the UV radiation exposure monitoring apparatus 104 identifies any health-related factors impacting a user's 400 UV exposure threshold, as described in more detail below.

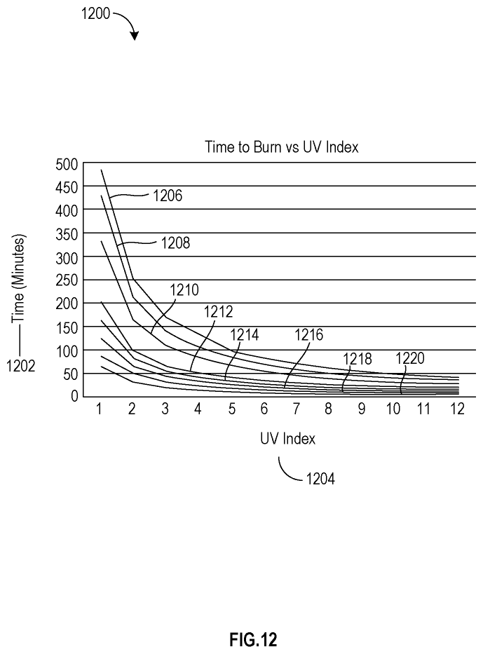

In some embodiments, the UV radiation exposure monitoring apparatus 104 may present to the user 400 queries 906 related to occupation. For example, if a user 400 400 provides input 908a-908d indicating participation in an occupation that requires work outside such as construction, field work, gardening, or any other outdoor labor or occupation, this information may be factored into a maximum UV radiation exposure threshold 1200 for the user 400, as discussed in more detail with reference to FIG. 12 below.

In some embodiments, the physical characteristic input 908a-908d is used to determine the user's 400 Fitzpatrick Skin Type, a recognized dermatological scale used to assess an individual's photosensitivity and erythemal qualities. In one embodiment, the questionnaire 904 includes a series of ten (10) queries 906 regarding the user's 400 physical attributes and previous experience with sun exposure or overexposure that will allow an application-housed algorithm to determine the user's 400 Fitzpatrick Skin Type. For example, the user 400 may be asked to identify their eye color from a range of visual input 908a-908d options. Another query 906 might ask the user to identify the severity of their previous sunburns and results therefrom. Input 908a-908d provided in response to the questionnaire 904 may indicate a Fitzpatrick Skin Type that ranges from “Skin Type 1”, corresponding to the lightest and most potentially photosensitive skin, to “Skin Type 6”, corresponding to the most melanated and least potentially photosensitive skin. In some embodiments, this scale is altered to include half-step skin types for Types 1-3 to account for users 400 who are in-between skin types, thereby allowing for a more accurate assessment of a user's maximum UV exposure threshold.

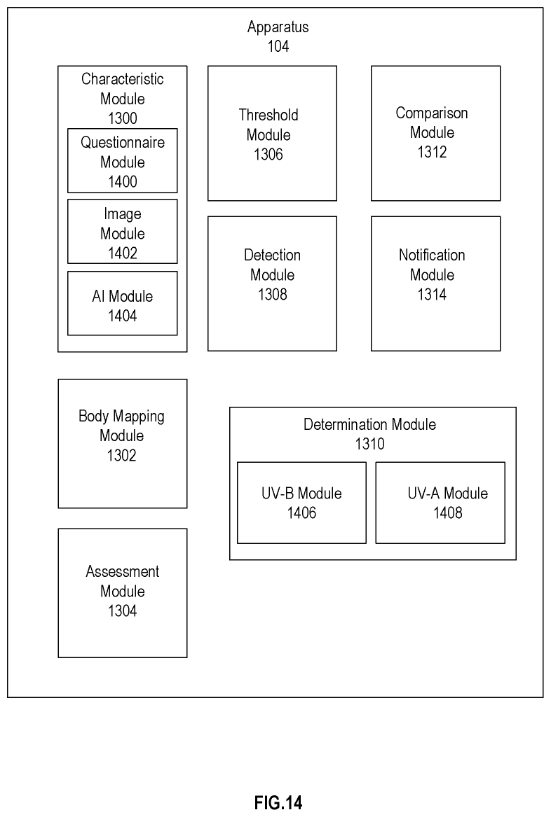

In some embodiments, the queries 906 allow the user 400 to choose from multiple visual input 908a-908d options, thereby increasing a likelihood of obtaining accurate results. Each question-and-answer combination may be separately weighted to achieve a photosensitivity score for the user 400. In some embodiments, a Fitzpatrick Skin Type is assigned to the user 400 based on this score. The results of the questionnaire 904 may be further enforced using an artificial intelligence (AI) component or module 1404, as discussed in more detail with reference to FIG. 14 below.

Referring now to FIG. 14 while still referring to FIGS. 11 and 1, for example, in some embodiments, the UV radiation exposure monitoring apparatus 104 is configured to provide solutions to potential “human error” that may occur when determining the user's 400 Fitzpatrick Skin Type. For example, if the user 400 encounters a query 906 that asks them to determine the natural color of their hair or eyes and they are unsure how to answer due to a limited range of options available, or false understanding of the true hue of the feature, the UV radiation exposure monitoring apparatus 104 may utilize the AI module 1404 to inform and/or reinforce the results of the input 908a-908d for determining the user's 400 Fitzpatrick Skin Type.

In some embodiments, the AI module 1404 may request additional imaging or scanning of the user's 400 face since the human face and neck are known to be the most photosensitive regions of the integumentary system. In some embodiments, the AI module 1404 is configured to analyze key physical attributes of the user 400, including, but not limited to, the user's 400 skin color (non-exposed), eye color, the user's 400 tendency to freckle, and more. In some embodiments, the AI module 1404 is implemented following completion of the questionnaire 904 and may require prior consent from the user 400. Upon completion of the facial scan, the data received may be quantified and corroborated with the data received from the user's 400 completion of the Fitzpatrick Skin Type questionnaire 904. In the event additional information is needed from the user 400, the UV radiation exposure monitoring apparatus 104 may present the user 400 with further questions and/or the AI module 1404 may request additional imaging and/or facial scan reinforcement, for example.

In some embodiments, the UV radiation exposure monitoring apparatus 104 is configured to receive from the wearable UV sensor assembly 102 current UV radiation level input including a current UV radiation level and an exposure time corresponding to at least one area 804 of the user's 400 body 802. In some embodiments, the UV radiation exposure monitoring apparatus 104 generates body mapping data 800 for the user 400 based on the physical characteristic input 908a-908d and/or the current UV radiation level input 908a-908d, as discussed above with reference to FIG. 8.

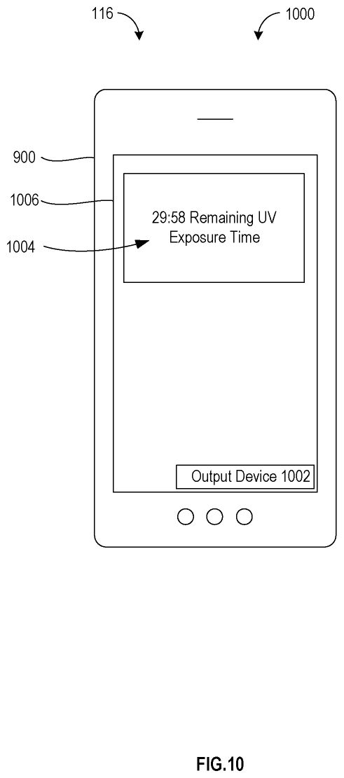

Referring now to FIG. 10, while still referring to FIGS. 1, 8, and 9, in some embodiments, the processor 108 of the UV radiation exposure monitoring apparatus 104 is configured to assess a UV exposure risk for each of area 804 of the user's 400 body 802 based on the physical characteristic input 908a-908d, the current radiation level input 908a-908d, and the body mapping data 800. The UV radiation exposure monitoring apparatus 104 may be further configured to determine, based on the UV exposure risk, a threshold UV radiation exposure value for each of the plurality of areas 804. A current UV radiation exposure value for the at least one area 804 may be determined based on the current UV radiation level and the exposure time. In some embodiments, the current UV radiation exposure value is compared to the threshold UV radiation exposure value for the at least one area 804 to generate UV radiation exposure data based thereon.

In some embodiments, the UV radiation exposure monitoring apparatus 104 includes a notification system 1000 configured to notify the user 400 of a remaining time 1004 for safe UV radiation exposure based on the UV radiation exposure data. In some embodiments, the notification system 1000 is configured to display on the display element 900 the remaining time 1004 for safe UV radiation exposure for one or more areas 804 of the user's 400 body. In some embodiments, the notification system 1000 is configured to alert the user 400 to potential overexposure to UV radiation in one or more areas 804 via an output device 1002. In some embodiments, the output device 1002 may be configured to produce, for example, an auditory alarm, haptic feedback, a visual notification, and/or the like.

Referring now to FIG. 12, in some embodiments, the UV radiation exposure monitoring apparatus 104 determines a remaining time 1202 for safe UV radiation exposure based on solar index (SI) dose limits 1200, or maximum UV radiation exposure thresholds 1200, that prevent erythemal reddening based on a predetermined Fitzpatrick Skin Type. FIG. 12 illustrates a summary of data points from various studies and figures that have been interpolated and extrapolated to determine a maximum UV exposure time 1202 based on an established UV index value 1204.

In some embodiments, the maximum amount of time a user 400 can spend under a prolonged, constant level of UV radiation, or maximum UV exposure time 1202, can be represented by the equation MaxTime=C*SI^{a}, where MaxTime is the maximum time 1202 in minutes and C represents the maximum UV radiation exposure threshold 1200. This equation incorporates an extrapolated and dermatologist-verified variable for a Fitzpatrick Skin Type's specific erythemal reaction, multiplied by the current solar index dose limit 1202 (converted from energy readings) to the power of coefficient ‘a’.

The coefficient ‘a’ is approximately −1.0 for all skin types, while C depends on a determined Fitzpatrick Skin Type. Given that ‘a’ is approximately −1.0 for all skin types, the maximum time 1202 can be multiplied by the SI reported via one data transmission to calculate C, representing the UV dose limit 1200 for reddening or burning for each Fitzpatrick Skin Type. It should be noted that SI multiplied by time is directly related to Joules and the accumulation of SI multiplied by time is proportional to total UV Joules incident on the skin. The net result is an maximum UV radiation exposure threshold 1200, C, for each of the Fitzpatrick Skin Types. As shown in FIG. 12, a maximum UV radiation exposure threshold 1200 is highest at any UV index value 1204 for skin type 6 1206, with increasingly reduced UV index values 1204 from skin type 5 1208 to skin type 4 1210, to skin type 3 1212, to skin type 2.5 1214, to skin type 2 1216, to skin type 1.5 1218 to skin type 1 1220, where skin type 1 1220 includes the lowest maximum UV radiation exposure threshold 1200 at any UV index value 1204.

In some embodiments, although a user 400 is assigned a set skin type value (e.g., Skin Type 3 1212) on the Fitzpatrick scale, this is solely for the user 400 to have a better understanding of where they are represented on the scale. In some embodiments, the official skin type used by the UV radiation exposure monitoring apparatus 104 to configure the maximum UV radiation exposure thresholds 1200 may include something more similar to Skin Type 2.683, for example, such that the UV radiation exposure monitoring apparatus 104 is configured to determine a maximum UV radiation exposure threshold 1200 for the user 400 with extreme accuracy.

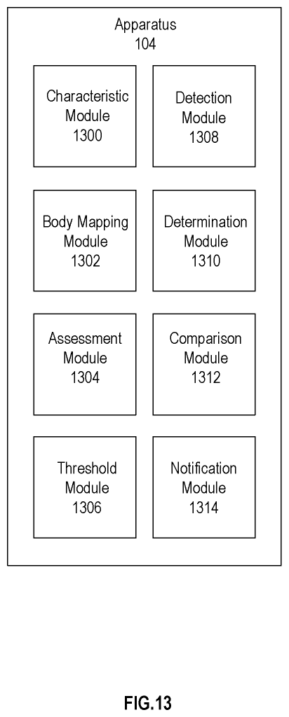

Referring now to FIG. 13, while also referring to FIG. 1, the UV radiation exposure monitoring apparatus 104 may include one or more processors 108 and non-transitory computer-readable storage media storing code executable by the processors 108 to perform operations. In some embodiments, the UV radiation exposure monitoring apparatus 104 includes a characteristic module 1300, a body mapping module 1302, an assessment module 1304, a threshold module 1306, a detection module 1308, a determination module 1310, a comparison module 1312, and a notification module 1314.

In some examples, the characteristic module 1300 is configured to receive input 908a-908d from the user 400. In some examples, the input 908a-908d includes one or more characteristics of the user 400. The characteristics include, for example, physical and/or genetic characteristics. In some examples, the characteristics include health and/or family history, such as a personal and/or family history of skin disease, sunburn, skin damage, and/or skin cancer. In some examples, the characteristics include prior activity of the user 400, such as previous sun exposure or a length of time that the user 400 has been exposed to sunlight that day prior to activating and/or utilizing the wearable UV sensor assembly 102. In some examples, the input 908a-908d includes input 908a-908d received as responses to queries 906 in a questionnaire 904 via a user input device 902, such as the inputs 908a-908d shown in FIG. 9. In some examples, the characteristics include characteristics used to determine the user's 400 Fitzpatrick Skin Type.

In some examples, the characteristics module 1300 is further configured to receive input 908a-908d regarding the user's 400 regular habits, including: habitual protective measures (e.g., sunscreen, covering, etc.), tanning bed use, level of SPF typically used, frequency of protective measures, medication use (e.g., medications that impact photosensitivity), medical and/or cosmetic procedures (e.g., procedures that impact photosensitivity, such as facials or chemical peels), occupation (e.g., an indoor occupation vs. an outdoor occupation, such as agriculture and/or construction), and/or any combination thereof.

In some examples, the characteristics module 1300 is configured to store different profiles including different characteristics for different users 400. For example, the characteristics module 1300 may store a profile for a user 400 and an additional profile for a family member of the user 400, based on that family member's characteristics.

In some examples, the body mapping module 1302 is configured to receive and/or generate body mapping data 800 for the user 400. In some examples, the body mapping data 800 is generated by the apparatus 104 (e.g., by a processor 108 of a computing device 116). In some examples, the body mapping data 800 includes a susceptibility for damage due to UV exposure for skin in a particular area 804 and/or at a particular point of the user 400's body 802. In some examples, the body mapping data 800 includes a location of the wearable UV sensor assembly 102 on the user 400's body. FIG. 8 illustrates examples of body mapping data 800 for particular users 400a, 400b, 400c. In some examples, the body mapping data 800 includes a risk level that is adjusted and/or generated based at least in part on apparel and/or other protective measures taken by a user 400 in that particular area of their body 802, such as a head covering 700, sunscreen, clothing, and/or any combination thereof.

In some examples, the body mapping data 800 is generated based at least in part on images of the user 400, answers to queries 906, other input 908a-908d from the user 400, and/or any combination thereof. In some examples, the input 908a-908d of the user 400 includes a location of previous skin damage, and the body mapping module 1302 is configured to adjust the body mapping data 800 accordingly. In some examples, the body mapping data 800 includes and/or is generated based on input 908a-908d comprising a visual representation of susceptibility of certain areas 804, such as the visual representations shown in FIG. 8. The visual representation may include, for example, an image file.

In some examples, the assessment module 1304 is configured to assess a UV exposure risk for each of a plurality of areas 804 of the user's 400 body 802 based at least in part on the input 908a-908d received by the characteristics module 1300 and/or on the body mapping data 800 received and/or generated by the body mapping module 1302.

In some examples, the risk assessment module 1304 determines a risk level that is inversely proportional to a numbering of a user's 400 Fitzpatrick Skin type. For example, the characteristics module 1300 determines that a user 400 has type 1 skin (e.g., very pale, white skin, with green or blue eyes), and the risk assessment module 1304 determines that their risk level is high. In another example, the characteristics module 1300 determines that a user 400 has type 6 skin (e.g., dark skin, eyes, and hair), and the risk assessment module 1304 determines that the risk level is relatively low. In some examples, in determining a risk level for the user 400, the risk assessment module 1304 weighs skin color more heavily than other factors.

In some examples, the risk assessment module 1304 determines a different risk level for different areas 804 of the user 400's body 802, according to body mapping data 800. For example, the risk assessment module 1304 determines a low risk level for a foot region 816 of the body 802, particularly if that region 816 is covered by clothing and/or shoes. In some examples, risk assessment module 1304 determines a high risk level for another region 808 that is more exposed and/or tends to be more exposed, such as a user 400's face and/or neck.

In some examples, the risk assessment module 1304 is configured to determine risk levels for different areas of a body 802 via a training process. In a number of examples, the risk assessment module 1304 instructs the user 400 to position themselves in exposure to UV radiation and to place the UV light sensor 110 in communication with the risk assessment module 1304, such as one of the wearable UV sensor assembly 102, in a particular area 804 of their body 802 (e.g., area 808). In some examples, the risk assessment module 1304 then collects data from the UV light sensor 110. The risk assessment module 1304 is configured to then, in some examples, instruct the user 400 to place the UV light sensor 110 on a different area 804 of their body 802 (e.g., area 810). In some examples, the risk assessment module 1304 collects data from the UV light sensor 110 after the location of the UV light sensor 110 is changed. In some examples, the risk assessment module 1304 is configured to determine relative risks for the different areas 804 (e.g., areas 808 and 810) of the body 802 based on the differences in UV radiation measured by the UV light sensor/(s) 110 when placed on those areas 804.

In some examples, the risk assessment module 1304 is configured to assign risk categories to the different areas 804 of the body 802 (e.g., “low risk”, “moderate risk”, and “high risk”). In some examples, the risk assessment module 1304 is configured to assign the areas 804 of the body 802 a numerical risk value (e.g., a percentage chance of sun damage in a given window of time and/or a numerical value on a scale of 1 to 100).

In some examples, the threshold module 1306 is configured to determine, based at least in part on the UV exposure risk, a threshold UV radiation exposure value for each of the plurality of areas 804. In some examples, the threshold UV radiation exposure is maximum a level of UV radiation exposure which the threshold module 1306 predicts that a particular user 400 will tolerate without significant damage (e.g., sunburn, heat sickness, etc.).

In some examples, the threshold is inversely proportional to the risk determined by the risk assessment module 1304. For example, the risk assessment module 1304 determines that a particular area 808 of a user's 400 body 802 is at high risk due at least in part to lack of coverage in that area 808 and/or a user's 400 characteristics. In such examples, the threshold module 1306 determines a lower threshold value in that area 808 than in another area 816 that benefits from more coverage and thus has a lower risk value, as determined by the risk assessment module 1304.

In some examples, the threshold value for an area 804 in which the wearable UV sensor assembly 102 is located is proportional to the risk level for that area 804. For example, a sensor assembly 102b located in a high-exposure area, such as a user's chest and/or neck area 808, is likely to detect a higher percentage of the UV radiation that a user's body 802 is being exposed to than a sensor assembly 102 located in a lower-exposure area, such as on a user's feet 816. As such, the threshold value for the higher-risk area 808 is higher than the threshold value for the lower-risk area 816, since the threshold value for the lower-risk area 816 is not as accurate of an indicator of total UV exposure for the user 400 and merits more caution when using it as a generalized indicator of UV exposure.

In some examples, the threshold is in the units of Joules (“J”) and/or millijoules (“mJ”). In some examples, the threshold for a particular skin type is proportional to a “time to burn” for that particular skin type, as shown in FIG. 12.

In some examples, the threshold module 1306 is configured to receive a skin type from the characteristics module 1300 and/or determine a skin type based on information from the characteristics module 1300. In some examples, the threshold module 1306 stores baseline threshold values for each of a number of skin types (e.g., each of the Fitzpatrick skin types) and is configured to determine the threshold UV radiation level based at least in part on the skin type. In some examples, the threshold module 1306 assigns a baseline threshold level of 1500-6000 mJ for users with more susceptible skin types. In some examples, the threshold module 1306 assigns a baseline threshold level of 800-1400 mJ for less susceptible skin types.

In some examples, the threshold module 1306 is configured to determine the baseline threshold value for a particular area of the body 802 based at least in part on skin type and/or a risk level for that area 804 and to adjust the baseline threshold value based at least in part on acute characteristics received from the characteristics module 1300. Acute characteristics include mutable characteristic that may vary for a particular user 400 over time. Acute characteristics may include, for example, current skin damage, prior sun exposure, use of products applied to the skin (e.g., sunscreen, antioxidants, moisturizer, aloe vera etc.), clothing worn underneath the UV light sensor 110, other coverage for the skin positioned beneath the UV light sensor 110, and/or any combination thereof.

For example, in one embodiment, the characteristics module 1300 determines that a user 400 has fair skin. In response, the threshold module 1306 determines a baseline threshold value of 2000 mJ for that area of the user's 400 body 802. However, the threshold module 1306 determines that the user 400 has applied sunscreen to the relevant area(s) 804 and has a slight tan from moderate prior sun exposure but does not have any burns. In response, the threshold module 1306 increases the dose from the baseline threshold of 2000 mJ to a more accurate threshold value of approximately 2500 mJ, since the skin will not be exposed to all of the UV radiation picked up by the UV light sensor 110 in that area (i.e., due in part to the sunscreen blocking some of that radiation).

In some examples, the threshold value reflects an estimated maximum level of UV radiation that could be detected via a UV sensor placed on a particular portion of the body 802 without the user 400 experiencing skin damage, which may be higher than a level of UV radiation that would harm the user 400 without the presence of certain acute characteristics (e.g., sunscreen, clothing, etc.). In some examples, the threshold module 1306 weighs genetic factors received from the characteristics module 1300 (e.g., eye color, skin color, etc.) more heavily than other factors, such as acute characteristics and/or habits.

In some embodiments, the threshold module 1306 is further configured to account for a time-varying protective effect associated with products applied to the skin, including sunscreen. The time-varying protective effect may be based on an initial protection level corresponding to a sun protection factor (SPF) value and may decrease over time. Thus, in certain embodiments, the processor 108 applies a decay function to the initial protection level to determine a current effective protection level. The decay function may be based on elapsed time since application and may include, for example, a linear decay, exponential decay, stepwise reduction, or a learned decay model. In some embodiments, the decay of the protective effect is modified based on one or more inputs, including environmental conditions and user activity. Such inputs may include, but are not limited to, perspiration, water exposure, ambient temperature, humidity, and physical activity level.

In certain embodiments, the processor 108 is configured to distinguish between different sunscreen formulations, including chemical and mineral formulations for example, and applies different decay characteristics based on the formulation type. In some embodiments, the current effective protection level is incorporated into the determination of the threshold UV radiation exposure value, such that degradation of the protective effect results in a corresponding adjustment to the threshold value over time.

In certain embodiments, the processor 108 is configured to dynamically update the effective protection level during an exposure session and generate a notification or recommendation when the protection level falls below a threshold value. The notification may include, for example, recommending reapplication of sunscreen.

In some embodiments, the characteristics module 1300 and/or the threshold module 1306 are configured to maintain a dynamic representation of a user's skin state. The dynamic skin state may include one or more parameters indicative of the user's current sensitivity to UV radiation, including degree of tanning, presence of erythemal damage, and/or cumulative UV exposure. In certain embodiments, the dynamic skin state is updated over time based on one or more inputs, including detected UV exposure levels, duration of exposure, user-provided feedback, environmental conditions, and/or sensor-derived or image-based analysis of the user's skin.

In some embodiments, the dynamic skin state reflects both temporary and longer-term changes in the user's skin. Temporary changes may include, for example, recent sun exposure or minor skin irritation. Longer-term changes may include, for example, gradual tanning or changes in baseline sensitivity resulting from repeated exposure. In certain embodiments, the threshold module 1306 is configured to adjust the threshold UV radiation exposure value based at least in part on the dynamic skin state, such that changes in the user's skin condition over time result in corresponding adjustments to the threshold value.

In some embodiments, the processor 108 is configured to apply a weighting or decay function to prior exposure data when determining the dynamic skin state such that more recent exposures are weighted more heavily than older exposures. In certain embodiments, the dynamic skin state may be further refined using machine learning or heuristic models that correlate observed exposure patterns and user outcomes with changes in UV sensitivity, thereby improving the accuracy of threshold determination over time.

In some examples, the display element 900 displays a graphical user interface (“GUI”) with a “toggle” element for the user 400 to input acute characteristics. For example, if the user 400 has applied sunscreen prior to initializing the device or if the user is wearing protective gear prior to initializing the device, the user 400 could switch these options to “ON” via the toggle elements.

In some examples, the detection module 1308 is configured to detect UV radiation incident on the UV light sensor 110 associated with the user 400. In a number of examples, the detection module 1308 is configured to detect, via the UV light sensor 110 (for example, a wearable UV light sensor), a current UV radiation level and/or an exposure time corresponding to at least one of the plurality of areas 804 for which the threshold module 1306 determined a threshold value.

In some examples, the UV sensing assembly 102 sends UV detection data to the monitoring apparatus 104 in packets of data. In some examples, the detection module 1308 is configured to receive, accumulate, process, and/or store this data.

In some examples, the at least one area 804 is an area 804 in which the UV light sensor 110 is located. In some examples, the UV light sensor 110 is part of the wearable UV sensor assembly 102 that is configured to be worn by the user 400 at one or more areas 804 of their body 802. For example, as shown in FIG. 8, the wearable UV sensor assembly 102a is located in a forearm area 812. In some examples, the wearable UV sensor assembly 102b is located in a neck/upper chest area 806. In some examples, the wearable UV sensor assembly 102c is located on a helmet 700 worn by a user 400c. In some examples, the wearable UV sensor assembly 102 is located on an upper arm area of the user 400c.

In some examples, the detection module 1308 is communicative with the UV light sensor 110 and/or the UV radiation assembly 102. In some examples, the detection module 1308 is configured to detect the UV radiation at periodic intervals. In some examples, the detection module 1308 is configured to poll the UV light sensor 110 periodically. In some examples, the detection module 1308 is configured to poll the UV light sensor 110 at a period in a range between about thirty (30) seconds and about five (5) minutes.

In some examples, the detection module 1308 is configured to detect an exposure time for at least one area 804 of the body 802. In some examples, the exposure time includes a total length of time which has passed since an initial time beginning the period of UV exposure and the current time. In some examples, the initial time is determined based at least in part on input 908a-908d from the user 400. In some examples, the user 400 selects a “start” option displayed on the display element 900 via a user interface. In some examples, the user 400 inputs a time at which they began their sun exposure. In some examples, the initial time is a time of day at which the detection module 1308 first detects UV radiation via the UV light sensor 110.

In some examples, the detection module 1308 is configured to separately detect UV-A radiation and UV-B radiation. In some examples, the detection module 1308 is configured to update a visual display (e.g., via the display element 900) to show the detected radiation. In some examples, the detection module 1308 is configured to present a display showing a user 400's body 902, as shown in FIG. 9. In some examples, as the detection module 1308 receives data on UV exposure, the detection module 1308 continuously updates the display to reflect areas 804 of the user's body 902 that have been exposed to UV radiation. In some examples, updating the display includes darkening and/or otherwise changing a color of a portion of a visual representation of the user's 400 body 802 on the display 902. In some examples, the detection module 1308 updates the display 902 based at least in part on a type of UV radiation detected (e.g., UV-A and/or UV-B). Since UV-B radiation can be more immediately damaging to a user 400's skin, in some examples, the detection module 1308 weighs UV-B radiation more heavily when updating the display.

In some examples, based on the characteristics of the user 400, the monitoring apparatus 104 is configured to determine four levels of accumulation for the user, characterized as basic exposure, intermediate exposure, advanced exposure, and dangerous exposure. In some examples, on a visual display of the user's 400 body 802, these levels coordinate with colors to serve as visual indicators for the user 400 to understand their pathway to reaching maximum UV exposure levels. In some examples, the basic exposure region of a user's 400 identified maximum exposure range coordinates with the color green, intermediate exposure with the color light orange, advanced exposure coordinates with the color red, and dangerous exposure coordinates with the color black. In some examples, these visual indicators can help the user to understand what level of UV exposure they are currently in, based on accumulated UV detection.

In some examples, the determination module 1310 is configured to determine a current UV radiation exposure value for at least one of the plurality of areas 804 of the user's 400 body 802 based on at least one of the current UV radiation level and/or the exposure time received from the detection module 1308. In some examples, the current UV radiation exposure value reflects a total UV exposure for a particular area 804 of the body 802 over a time period commencing at the initial time. In some examples, the total UV exposure is determined based on an aggregation of one or more measurements received from the UV light sensor 110. In some examples, the total UV exposure is determined based on the highest measurement received from the UV light sensor 110. In some examples, the total UV exposure is determined based at least in part on measurements from multiple UV sensors positioned on different portions of the body 802. In some examples, the determination module 1310 determines the total UV exposure to be an area 804 under a curve of a graph of UV detection over time.

In some examples, the comparison module 1312 is configured to compare the level of UV exposure determined by the determination module 1310 to the threshold UV radiation exposure value determined by the threshold module 1306. In some examples, the level of UV exposure is determined for a particular area 804 of the body 802 and compared to a threshold value for that area 804.

In some examples, the comparison module 1312 is configured to determine a difference between the current UV radiation exposure value and the threshold UV radiation exposure value. In some examples, the comparison module 1312 is configured to determine whether the current UV radiation exposure value is within a given range of the threshold UV exposure value.

In some examples, the comparison module 1312 is configured to determine that the current UV radiation exposure value is less than the threshold UV exposure value and to determine a remaining time before the UV radiation exposure value is predicted to meet and/or exceed the threshold value. In some examples, the comparison module 1312 predicts the remaining time based at least in part on past measurements from the UV light sensor 110, times of past measurements from the UV light sensor 110, characteristics from the characteristic module 1300, acute characteristics, and/or any combination thereof.

In some examples, the comparison module 1312 is configured to predict a remaining time based at least in part on a relationship between a total safe time for UV exposure, a skin type, and a UV index for the user's location, such as those shown in FIG. 12. In some examples, the comparison module 1312 is configured to communicate with weather information provider (e.g., via an application programming interface (“API”)) to determine the UV index for the user's 400 location.

In some examples, the comparison module 1312 is configured to generate UV radiation exposure data. In some examples, the UV radiation exposure data includes the current UV radiation exposure and/or a predicted remaining time for safe UV radiation exposure.

In some examples, the notification module 1314 is configured to notify the user 400 of the remaining time for UV radiation exposure based on the UV radiation exposure data. In some examples, the notification module 1314 is configured to notify the user 400 via the computing device 116. In some examples, the notification includes at least one of haptic feedback (e.g., vibrations), sound, a change and/or addition to the display element 900, and/or any combination thereof. In some examples, the notification is similar to the notification shown in FIG. 10.

In some examples, the threshold module 1306 is configured to determine four levels of threshold accumulation for the user 400, characterized as basic exposure, intermediate exposure, advanced exposure, and dangerous exposure. In some examples, the notification module 1314 is configured to notify the user 400 of exposure levels at each threshold shift. In some examples, when the user's 400 exposure accumulation status has surpassed the basic exposure range and has entered the intermediate exposure range, the notification module 1314 notifies the user 400. In some examples, the notification module 1314 is configured to determine a notification frequency based at least in part on changes in the remaining time. In some examples, notification frequency is customizable by the user 400. In some examples, the notification module 1314 is configured to notify the user 400 more frequently given the amount of time adjusted to remain for the user's 400 activity.

In some examples, the notification module 1314 is configured to use methods of machine learning to find the ideal moments during a user's 400 UV exposure session to notify them. In some examples, the notification module 1314 is configured to, after a UV exposure session has ended, display a graph summarizing actual UV exposure over time during the period of use based at least in part on measurements received from the detection module 1308.

In some examples, the UV radiation exposure monitoring apparatus 104 is configured to enable a user 400 to select points of time along that graph at which they determined they may have felt at risk of developing sun damage. In some examples, the UV radiation exposure monitoring apparatus 104 is configured to further enforce these inputs by asking a user 400 post-activity-questions, such as if the user 400 experienced any erythemal reddening following use of the UV light sensor 110, when the reddening occurred and where, as well as additional qualitative and quantitative inquiries. In some examples, the threshold module 1306 is configured to adjust the user's 400 maximum exposure threshold based at least in part on these inputs. In some examples, the notification module 1314 is configured to adjust the frequency in which the user 400 is notified based at least in part on these inputs. In some examples, the UV radiation exposure monitoring apparatus 104 is configured to store these inputs over time for multiple sessions of UV exposure and to employ a machine learning method to determine threshold levels and/or how frequently to notify a user 400.

Referring now to FIG. 14, in some embodiments, the UV radiation exposure monitoring apparatus 104 further includes a questionnaire module 1400, an image module 1402, an AI module 1404, a UV-B module 1406, and a UV-A module 1408.

In some examples, the characteristic module 1300 includes the questionnaire module 1400. In some examples, the questionnaire module 1400 is configured to generate a questionnaire for the user 400 regarding their physical characteristics, as shown in FIG. 9. In some examples, the input 908a-908d received by the characteristics module 1300 includes input 908a-908d received from the user 400 in response to the questionnaire 904. In some examples, the questionnaire 904 includes at least one of a user's 400 hair color, a user's 400 skin color, a user's 400 eye color, the presence of freckles, how the user's 400 skin typically reacts to sun exposure (e.g., burning, blistering, tanning), the degree to which the user 400 tans (e.g., never tans to tans deeply), how sensitive the user's 400 face is to the sun, the amount of recent sun exposure the user 400 has had, whether they intentionally tan or expose themselves to sun for cosmetic reasons, and/or any combination thereof.

In some examples, the characteristic module 1300 includes the image module 1402. In some examples, the input 908a-908d received by the characteristic module 1300 includes an image of at least one physical characteristic of the user 400. As shown in FIG. 11, in some examples, the image is an image of the user's 400 face. In some examples, the image is taken via a camera of the input device 902 and/or uploaded from the input device 902.

In some examples, the image module 1402 is configured to analyze the image to determine a skin type for the user 400. In some examples, the image module 1402 is configured to derive characteristics from the image (e.g., eye color, skin color, hair color).

In some examples, the characteristic module 1300 includes an artificial intelligence (AI) module 1404. In some examples, the AI module 1404 is configured to analyze an image received by the image module 1402 to determine characteristics of the user 400. In some examples, the AI module 1404 is configured to use techniques such as Convolutional Neural Networks (CNNs), Support Vector Machines (SVMs), Local Binary Patterns (LBPs), and/or any combination thereof. In some examples, the AI module 1404 trained on training data that includes various images of different users 400 with different skin types and characteristics. In some examples, the AI module 1404 determines a skin type for the user 400 by analyzing the image of the user 400. In some examples, the AI module 1404 is further configured to determine a threshold UV exposure for the user 400 based on the image. In some examples, the AI module 1404 is trained on images of other users 400 and threshold UV exposures for those users 400. In some examples, the threshold module 1306 determines the threshold for the user 400 based at least in part on input from the AI module 1404.

In some examples, the AI module 1404 compares the features of the user 400 against a database of known characteristics associated with different skin types and/or levels of photosensitivity. In some examples, this database is a training database.

In some examples, the AI module 1404 scans a portion of the user's 400 body 802, such as their face, in addition to or in alternative to capturing an image of the user 400. Examples of inputs used by the AI module 1404 to determine a skin type include, but are not limited to skin color, eye color, freckles, and/or any combination thereof.

In some examples, the AI module 1404 is configured to correlate collected data from the scan and/or image with responses to the user's 400 questionnaire 904. In some examples, the AI module 1404 is configured to generate additional queries 906. In some examples, the characteristics module 1300 is configured to determine discrepancies between answers to queries 906 and features detected by the AI module 1404 and to update user characteristics based at least in part on input from the AI module 1404.

In some examples, the AI module 1404 is configured to determine a current level of skin damage, and the threshold module 1306 is configured to adjust (e.g., lower) the threshold based at least in part on this skin damage. In some examples, the AI module 1404 is configured to prompt the user 400 to scan their face before sun exposure and after sun exposure. In various examples, the AI module 1404 is configured to determine changes in the user's 400 features by comparing these scans. In some examples, the AI module 1404 detects skin damage between the before and after scans, and the threshold module 1306 uses this skin damage and the threshold value used during that period of exposure to update a machine learning module for determining the threshold value.

In some examples, the AI module 1404 is trained to detect skin damage and/or other dermatological features based on training data that includes images of dermatological features and/or skin damage. Such dermatological features include, for example: moles, freckles, skin texture, scars, signs of previous sun damage, masses, and/or any combination thereof. In some examples, the AI module 1404 is configured to recognize variations in skin condition by analyzing multiple images and/or scans of the user's 400 skin over time. In some examples, the monitoring apparatus 104 is configured to present such changes in a customized report.

In some examples, the AI module 1404 is configured to use protective measures (e.g., acute characteristics) employed by the user 400 and the variations in skin condition as inputs in a machine learning algorithm. In some examples, the AI module 1404 is configured to use this machine learning algorithm to make recommendations for particular users 400 (e.g., protective coverings, SPF use, etc.) to help mitigate these variations in skin conditions. In some examples, the AI module continuously learns about the user 400 over time and over multiple sessions of UV exposure to provide tailored recommendations for that user 400. In some examples, the AI module 1404 is configured to recommend placement of the sensor assembly 102.

In some examples, the determination module 1310 includes a UV-B module 1406 and/or a UV-A module 1408. In some examples, the determination module 1310 is configured to determine a current level of UV-A exposure and a current level of UV-B exposure. In some examples, the determination module 1310 is configured to modify the total level of current UV exposure by weighing UV-A or UV-B more heavily than the other. In some examples, the determination module 1310 operates under a short-term skin health setting and weighs UV-B more heavily than UV-A, since UV-B is often more likely to cause short-term skin damage, such as sun burns.

In some examples, the determination module 1310 operates under a long-term skin health setting and weighs UV-A more heavily than UV-B, since UV-A rays can penetrate the skin more deeply and may have longer-lasting effects than UV-B rays. In some examples, the UV radiation exposure monitoring apparatus 104 receives input from the user 400 regarding a preference for short term skin health, long term skin health, or both. In some examples, the UV radiation exposure monitoring apparatus 104 receives a preference for short term skin health, and the determination module 1310 weighs the UV-B radiation from the UV-B module 1406 more heavily than the UV-A radiation from the UV-A module 1408. In various examples, the UV radiation exposure monitoring apparatus 104 does not receive a preference from the user 400 and defaults to weighing the UV-B radiation more heavily. In some examples, the UV radiation exposure monitoring apparatus 104 assigns the weights based at least in part on at least one of a family history of skin disease, a personal health history, characteristics from the characteristics module 1300, and/or any combination thereof. For example, if the user 400 indicates that they have a family history of skin cancer, in some examples, the determination module 1310 weighs the UV-A radiation more heavily than the UV-B radiation. In some examples, the UV radiation exposure monitoring apparatus 104 receives input from the user 400 that includes a percentage (e.g., 60% long-term skin health and 40% short-term skin health), and the determination module 1310 weighs the total determined UV-A radiation from the UV-A module 1408 and the total determined UV-A radiation from the UV-B module based at least in part on this percentage. As such, in some examples, the determination module 1310 determines a total UV exposure that is different from the user's 400 actual total UV exposure during that period due to the weighing of different types of UV radiation.

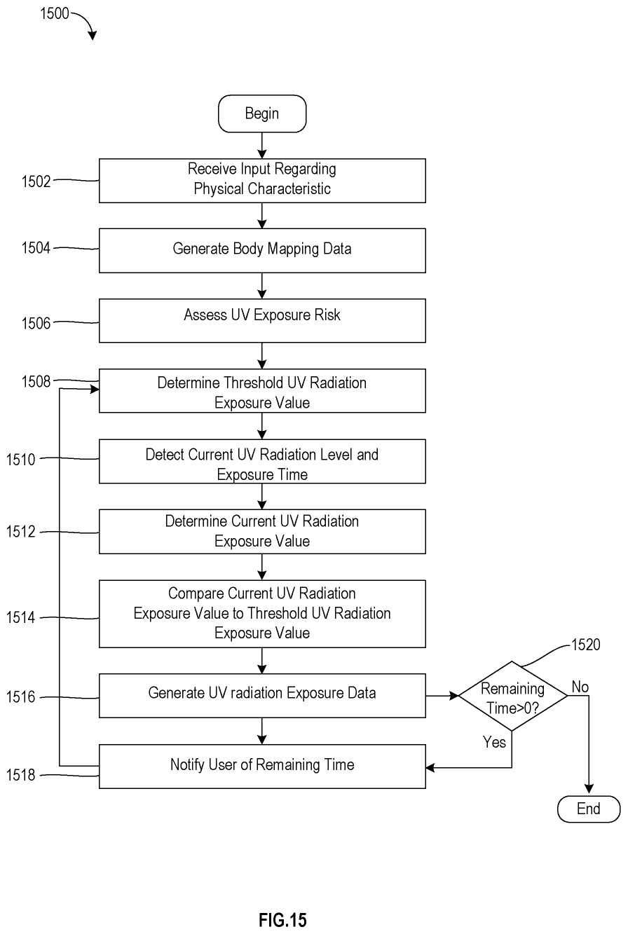

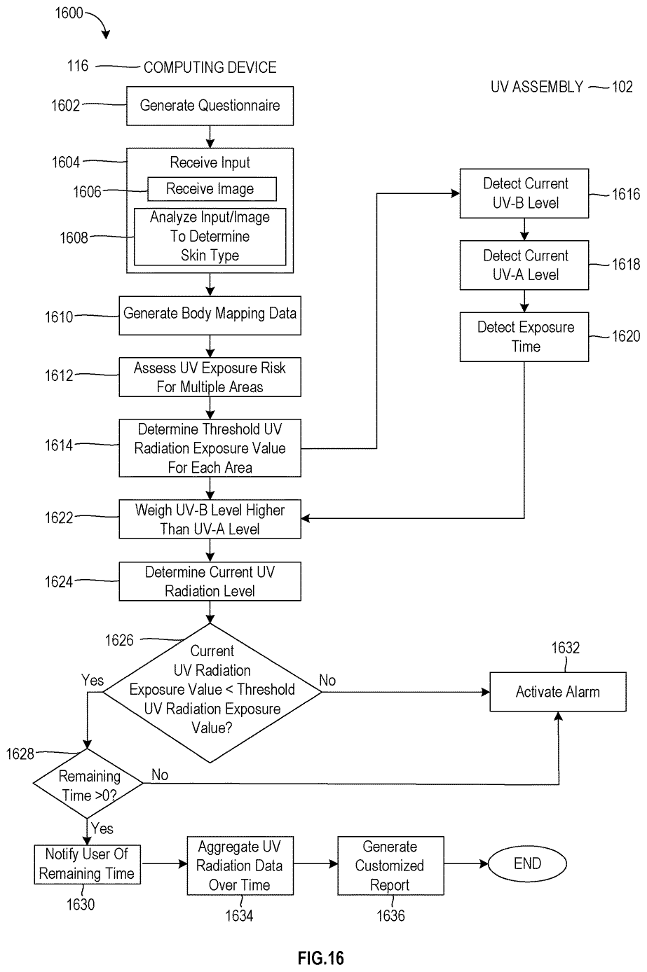

Referring now to FIGS. 15 and 16, a method 1500 for monitoring UV radiation exposure and notifying a user 400 of a risk of overexposure includes receiving 1502, by a processor 108, input 908a-908d including at least one physical characteristic of a user. The method 1500 further includes generating 1504, by the processor 108, body mapping data 800 for the user and assessing 1506, by the processor 108, a UV exposure risk for each of a plurality of areas 804 of the user's body 802 based on the input 908a-908d and the body mapping data 800.

The operations further include determining 1508, based on the UV exposure risk, a threshold UV radiation exposure value for each of the areas 804. In some examples, the method 1500 includes detecting 1510, via the UV light sensor 110, a current UV radiation level and exposure time for the at least one of the areas 804. In some examples, the method 1500 includes determining 1512 a current UV radiation exposure value based at least in part on the detecting 1510. In some examples, the method 1500 includes comparing 1514, by the processor 108, the current UV radiation exposure value to the threshold UV radiation exposure value for one or more of the areas 804 and generating 1516 UV radiation exposure data based thereon. In some examples, the method 1500 includes notifying 1518, by the processor 108, the user 400 of a remaining time for UV radiation exposure of the area 804 based on the UV radiation exposure data.

The method 1500 includes determining 1508, based on the UV exposure risk, a threshold UV radiation exposure value for each of the plurality of areas 804. A wearable ultraviolet (UV) light sensor assembly 102 is configured to be worn by the user in at least one of the areas 804 and is used to detect 1510 a current UV radiation level and an exposure time. The exposure time indicates an amount of time that the area 804 has been exposed to UV radiation. The method 1500 further includes determining 1512, by the processor, a current UV radiation exposure value for the area 804 based on the current UV radiation level and the exposure time. The processor 108 compares 1514 the current UV radiation exposure value to the threshold UV radiation exposure value for the area to generate 1516 UV radiation exposure data, including a remaining time of safe UV exposure. The method the queries 1520 whether the remaining time is greater than zero. If yes, the processor 108 notifies the user 400 of the remaining time for UV radiation exposure based on the UV radiation exposure data and returns to detecting 1510 a current UV radiation level and an exposure time.

In some embodiments, the method 1500 includes iteratively detecting the current UV radiation exposure radiation level and the exposure time at predetermined periodic intervals and updating the remaining time for UV exposure based on the detection. In some examples, the method 1500 is performed by one or more of the modules of the UV radiation exposure monitoring apparatus 104.

Referring now to FIG. 16, in some embodiments, a method 1600 for monitoring UV radiation exposure and notifying a user 400 of a risk of overexposure is, in some examples, an embodiment of the method 1600. In some embodiments, some steps of the method 1600 may be performed by the computing device 116, while other steps of the method 1600 may be performed by the wearable UV sensor assembly 102.

In some embodiments, the method 1600 includes generating 1602 a questionnaire 904, as illustrated in FIG. 9, and receiving 1604 input 908a-908d from the user 400. In some examples, the input 908a-908d includes information provided in response to the questionnaire 904. In some examples, receiving 1604 the input 908a-908d includes receiving 1606 an image, as illustrated in FIG. 11. In some examples, the receiving 1604 the input 908a-908d includes analyzing the input 908a-908d and/or the image to determine a skin type for the user 400.

In some examples, the method 1600 includes generating 1610 body mapping data 800. In some examples, the method 1600 includes assessing 1612 a UV exposure risk for each of multiple areas 804 of a user 400's body 802. In some examples, the method 1600 includes determining 1614 a threshold UV radiation exposure value for each area 804 of the body 802. In some examples, steps 1602-1614 of the method 1600 are performed by a processor 108 of the computing device 116.

In some examples, the method 1600 includes detecting 1616, at the UV light sensor 110, a current UV-B level. In some examples, the method 1600 includes detecting, at the UV light sensor 110, a current UV-A level. In some examples, the method 1600 includes detecting 1620, at the UV light sensor 110, a total exposure time of the user 400 to UV radiation.

In some examples, the method 1600 includes weighing 1622, by a processor 108 of the computing device 116, the current UV-B level higher than the current UV-A level. In some examples, the method 1600 includes determining 1624 a current UV radiation level based at least in part on these detected values and weights.

In some examples, the method 1600 includes determining 1626 whether a current UV radiation exposure value is less than a threshold UV radiation exposure value. If yes, the method 1600 includes determining 1628 whether the time remaining for safe UV radiation exposure is greater than zero. If no, and/or the current UV radiation exposure value is equal to or greater than the threshold UV radiation exposure value, the method 1600 includes activating 1632 an alarm to alert the user 400 to a risk of overexposure. In some examples, activating 1632 the alarm includes activating an alarm of the wearable UV light assembly 102. In other examples, the alarm is activated at the computing device 116.

In some examples, if the time remaining for safe UV radiation exposure is greater than zero, the method 1600 includes notifying 1630 the user 400 of the remaining time. In some embodiments, the user 400 may be notified of the remaining time via the display element 900 of the computing device 116. In some examples, the method 1600 includes aggregating 1634 UV radiation data over time for that user 400 and/or generating 1636 a customized report for the user 400 based on the aggregate data. In some examples, the customized report includes at least one of a total exposure time, total UV radiation exposure, total UV-A radiation exposure, total UV-B radiation exposure, skin type, characteristics, body mapping data, a recommendation for protection from UV radiation exposure, and/or any combination thereof.

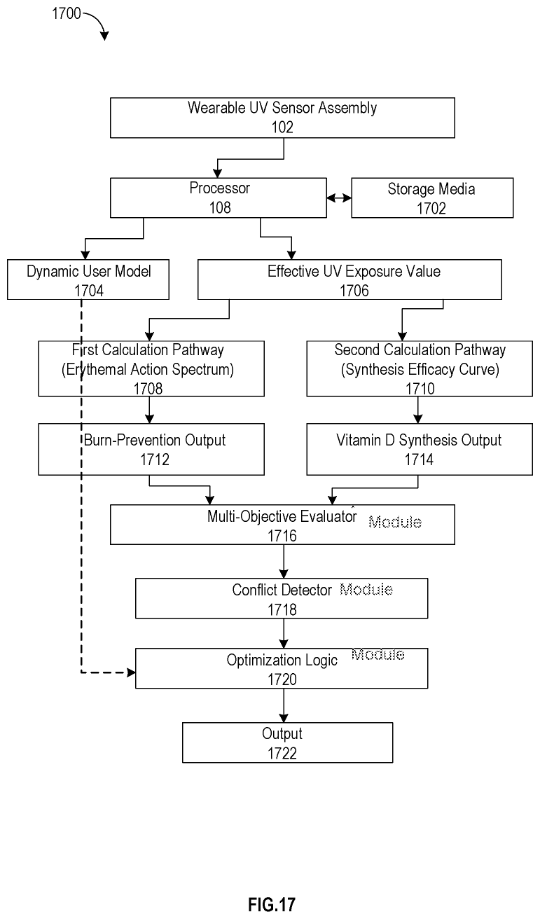

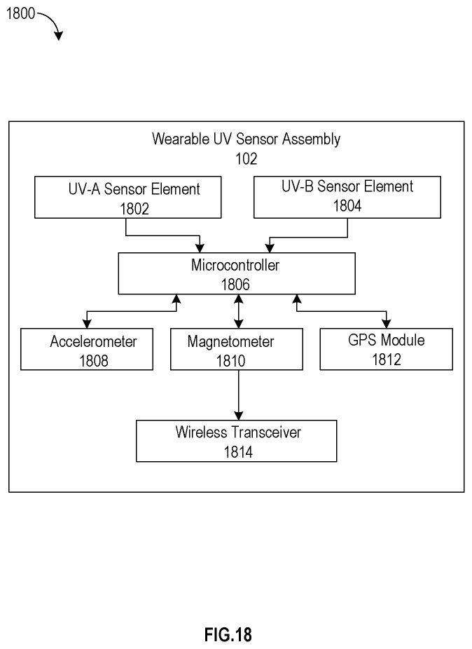

Referring now to FIG. 17, one embodiment of a system 1700 for dynamically managing ultraviolet (UV) radiation exposure of a user with simultaneous burn prevention and Vitamin D synthesis tracking capabilities is illustrated. The system 1700 may include a wearable UV sensor assembly 102 configured to detect UV radiation data associated with the user. In some embodiments, the wearable UV sensor assembly 102 may be substantially the same as that described below with reference to FIG. 18. The system 1700 may further include a processor 108 and storage media 1702 storing program code executable by the processor 108 to perform operations.

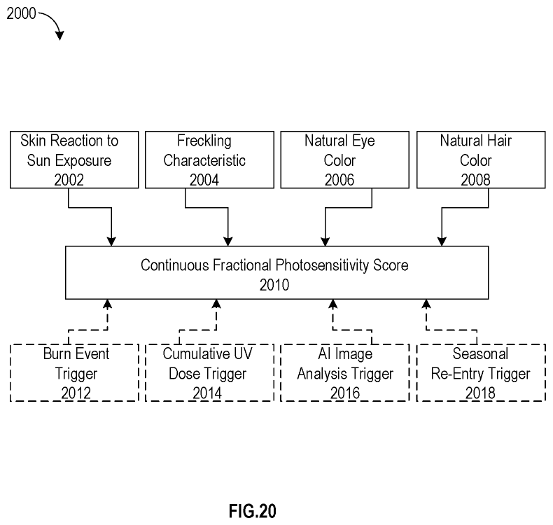

In some embodiments, the system 1700 is configured to maintain a dynamic user model 1704 associated with a user. The dynamic user model 1704 may be updateable over time based on input data and may represent a current response of the user to UV exposure. In some embodiments, the dynamic user model 1704 includes a continuous fractional photosensitivity score derived from a plurality of weighted inputs, as described in greater detail with reference to FIG. 20 below. The continuous fractional photosensitivity score may be used as a direct numerical input to dose calculations without rounding to a discrete integer value, thereby producing more precise UV exposure thresholds than discrete skin type assignment may allow. In some embodiments, the dynamic user model 1704 is updated based on one or more of a user-reported skin reaction, a cumulative UV dose accumulation crossing a defined level, an AI image analysis detecting visible changes in skin characteristics over time, and a seasonal re-entry prompt.

In some embodiments, the dynamic user model 1704 further includes a tanning indication input. A user-reported tanning response without burning may trigger a small upward adjustment to the continuous fractional photosensitivity score, thereby reflecting a modest increase in melanin-bases UV tolerance. The system 1700 may model a biological distinction between adaptive tanning, which produces a short-term modest tolerance increase, and cumulative photodamage, which produces a long-term irreversible threshold reduction. These two effects may operate as separate components in UV radiation dose calculation and may not be directly offset against each other.

In some embodiments, the dynamic user model 1704 maintains a longitudinal behavioral compliance model that governs the weight assigned to user-provided inputs in the threshold determination architecture. The compliance model may track user behavior access sessions and adjust input weighting based on observed adherence to system recommendations. Signals that indicate low user adherence and reduce the weight assigned to user-provided inputs may include, but are not limited to, ignoring burn-time notifications without subsequently reporting a skin reaction, self-adjusting dose upward repeatedly through a user override mechanism, reporting burns in sessions where the user had overridden the system-recommended threshold, and/or low sunscreen reapplication compliance as measured by the frequency with which reapplication notifications are acknowledged and reapplication events are confirmed.

When the compliance model determines that a user's self-reported inputs have low reliability for a specific input category, the system 1700 may apply a weighted blend between the user-reported input value and a more conservative system-calculated default for that input category. In some embodiments, the compliance model is updated using a machine learning process that analyzes at least one of notification response behavior, sunscreen compliance patterns, and user override frequency to adjust a reliability weight assigned to user-provided inputs. The compliance model output may be surfaced to clinicians in a clinician portal as a compliance score.

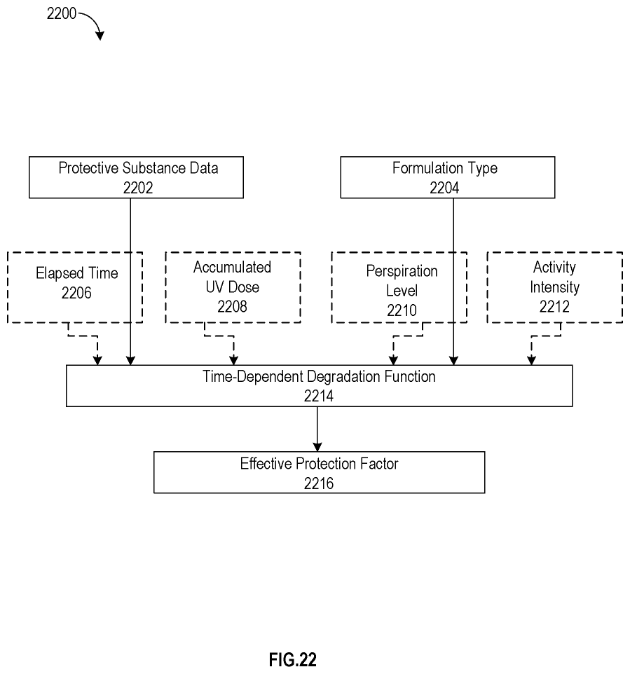

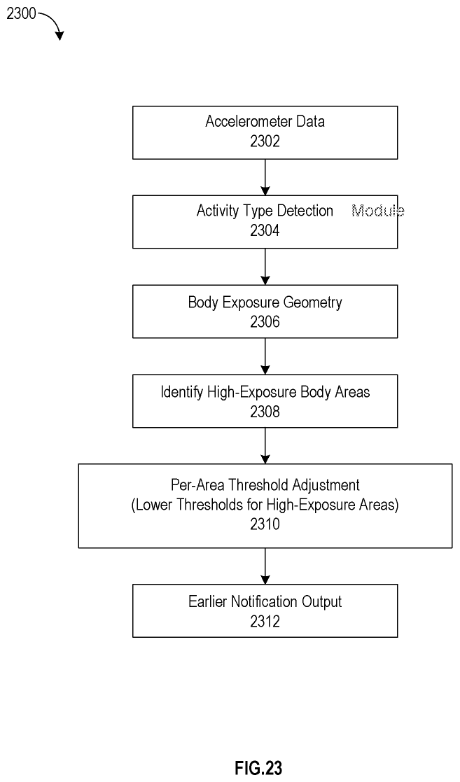

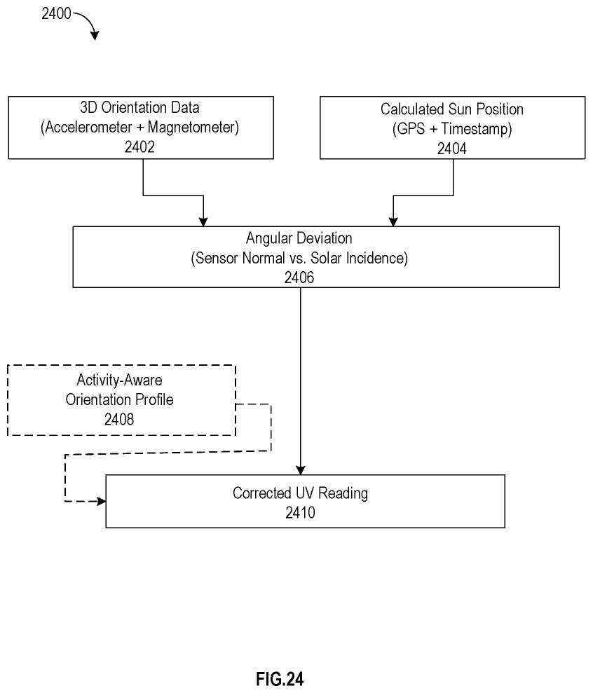

In some embodiments, the system 1700 determines an effective UV exposure value 1706 based on input data and at least one variable modification factor. The effective UV exposure value 1706 may account for factors including time-dependent protective substance degradation as described with reference to FIG. 22, activity-derived body exposure geometry as described with reference to FIG. 23, and/or orientation-based angular correction as described with reference to FIG. 24. In some embodiments, the effective UV exposure value 1706 is derived from independently measured UV-A radiation data and UV-B radiation data obtained from separate sensor elements of the wearable UV sensor assembly 102.

In certain embodiments, the effective UV exposure value 1706 is further modified by a user-configurable concern weighting that adjusts a relative contribution of the UV-A radiation data and the UV-B radiation data to the effective UV exposure value 1706. For example, in some embodiments, the user indicates the relative importance of skin cancer prevention and photoaging protection using a slider, scale, or multi-concern input interface in a companion app. The input may allow simultaneous weighting of both concerns on a continuous scale: for example, 70% skin cancer prevention and 30% photoaging protection. The weights need not sum to any specific value and may be adjusted independently.

In these and other embodiments, a user may weight a single concern exclusively. The concern weighting may be stored as a general profile setting and applied across all sessions by default. In some embodiments, the user configures activity-specific concern weightings that override the profile default for sessions of a particular activity type. Seasonal adjustment of concern weighting may be supported by some embodiments, reflecting the biological reality that UV-A levels remain relatively consistent year-round while UV-B levels peak in summer, making the relative clinical relevance of each band seasonally variable.

In some embodiments, the concern weighting directly modifies the per-area UV exposure threshold architecture by adjusting the relative contribution of UV-A and UV-B dose accumulation to the threshold consumption calculation. In the default system configuration, UV-B is weighted more heavily than UV-A in the dose determination, consistent with UV-B's dominant role in acute erythemal damage and skin cancer risk. When a user adjusts their concern weighting to increase the relative importance of photoaging protection, the system 1700 may be configured to increase the weight assigned to UV-A dose accumulation in the threshold consumption calculation to thereby produce earlier notifications when UV-A dose accumulation is high even if UV-B dose remains within the standard threshold. The concern weighting may not override the photosensitivity-based baseline threshold or any clinician-set threshold modifications. Instead it may operate as a weighting modifier within the multi-input threshold determination architecture, subject to the same conservative resolution rules governing that architecture.

In some embodiments, the concern weighting may influence notification content and framing. Notifications delivered to a user with a high photoaging weighting, for example, may reference UV-A dose accumulation explicitly and contextualize the notification in terms of aging risk rather than exclusively burn risk. Notifications delivered to a user with a high skin cancer prevention weighting, on the other hand, may reference UV-B dose accumulation and burn risk as the primary notification context. In some embodiments, the notification system delivers separate parallel notifications for UV-A and UV-B threshold proximity, thereby allowing the user to understand their status relative to each concern independently.

In some embodiments, reports generated for a user with active concern weighting may present UV-A and UV-B dose data as separate tracked metrics with independent trend analysis. The reporting content may emphasize the metrics most relevant to the user's stated concern weighting. In the clinical context, a clinician may review and modify a patient's concern weighting through the clinician portal if the clinical presentation warrants a specific emphasis. A clinician-set concern weighting may be configured to supersede the user's profile setting for the duration of the clinical relationship unless the clinician explicitly returns authority over the setting to the patient.

In some embodiments, the effective UV exposure value 1706 is further modified by environmental data inputs. For example, air quality data, including particulate matter concentration, pollution levels, aerosols, and atmospheric haze, may be sourced from environmental monitoring APIs or onboard air quality sensors and applied as an atmospheric attenuation correction to UV dose calculations. Where air quality data indicates high particulate concentration or pollution levels, the system 1700 may determine that the ambient UV reaching the user is lower than the UV index alone would suggest, and may adjust dose calculations accordingly.

Conversely, in environments where reflective surfaces, altitude, or atmospheric conditions amplify UV relative to the standard UV index, the environmental data inputs may support an upward correction. Temperature and humidity data, sourced from onboard environmental sensors, companion smartphone sensors, or weather API data, may serve as inputs to the dynamic SPF decay model described with reference to FIG. 22, where they function as proxies for perspiration rate and environmental conditions affecting sunscreen efficacy. Environmental pollutants and airborne chemical compounds known to interact with UV radiation at the skin surface may produce photochemical reactions that increase UV sensitivity or accelerate UV-induced skin damage. In some embodiments, these pollutants and compounds may be identified and applied as sensitivity modifiers to the user's UV exposure threshold for the current session.

In some embodiments, the effective UV exposure value 1706 is further modified by genetic risk inputs. Genetic variant data relevant to UV sensitivity and skin cancer risk, including but not limited to MC1R variants associated with red hair phenotype and elevated melanoma and non-melanoma skin cancer risk, CDKN2A mutations associated with familial melanoma susceptibility, and BRCA2 variants with established associations with melanoma risk, may be incorporated into the system 1700 to modify the risk threshold. Genetic data may enter the system 1700 through one or more of the following mechanisms: the user uploads a raw genetic data file from a consumer genomic testing service directly through a companion app or patient portal; the system 1700 imports genetic data through a structured API integration with a genomic testing partner; a clinician enters relevant genetic findings through the clinician portal; and/or the system 1700 partners with or provides access to a genetic testing service enabling users to complete genetic testing with results transmitted directly to the system.

In some embodiments, genetic data is processed transiently by the system 1700. Raw genetic data files or variant data transmitted to the system 1700 may be analyzed to identify relevant variants and generate a genetic risk modifier value, after which the raw genetic data may not be retained within the system 1700. Only the derived risk modifier value and the variant categories identified as relevant may be stored in connection with the user's health profile.

In some embodiments, identified genetic variants associated with elevated UV sensitivity or skin cancer risk are applied as modifiers within the multi-input threshold determination architecture, thereby reducing the user's per-area UV exposure threshold in proportion to the clinical significance of the identified variants. Genetic variants with established associations with skin photosensitivity, particularly MC1R variants that influence melanin production and UV response, may also be incorporated as inputs to the continuous fractional photosensitivity score described with reference to FIG. 20 to modify the continuous score directly.

In some embodiments, the effective UV exposure value 1706 is processed through a first calculation pathway 1708 and a second calculation pathway 1710 simultaneously. The first calculation pathway 1708 may apply an erythemal action spectrum to the effective UV exposure value 1706 to produce a burn-prevention output 1712. The erythemal action spectrum weights UV-B radiation more heavily than UV-A radiation in accordance with the relative contribution of each wavelength band to acute erythemal skin damage. In some embodiments, the burn-prevention output 1712 represents a remaining safe UV exposure time before erythemal threshold breach.

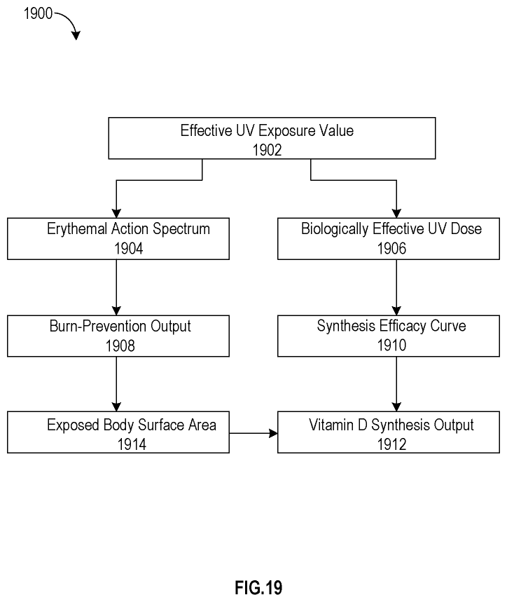

In some embodiments, the second calculation pathway 1710 applies a synthesis efficacy curve to the effective UV exposure value 1706 to produce a Vitamin D synthesis output 1714. The synthesis efficacy curve may be distinct from the erythemal action spectrum and may encode the relative effectiveness of each UV-B wavelength in driving cutaneous Vitamin D synthesis. In some embodiments, the synthesis efficacy curve reflects peak effectiveness at approximately 295 to 300 nanometers and declining efficacy at wavelengths above about 310 nanometers. In some embodiments, the second calculation pathway 1710 determines a biologically effective UV dose based on spectral components and combines this with an exposed body surface area to produce the Vitamin D synthesis output 1714, as described in greater detail with reference to FIG. 19. The Vitamin D synthesis output 1714 may be expressed in international units (IU) per session. In some embodiments, the system 1700 is configured to accumulate Vitamin D synthesis output 1714 at each polling interval as a function of all active modifying inputs.

In some embodiments, the second calculation pathway 1710 applies a synthesis-specific SPF attenuation model configured to operate in parallel to the burn-prevention SPF model described with reference to FIG. 22. The synthesis-specific SPF attenuation model may be configured to apply SPF attenuation specifically to the synthesis-effective UV-B fraction. Because synthesis-effective wavelengths are concentrated at the lower end of the UV-B spectrum (approximately 295 to 300 nanometers) and SPF ratings are defined in relation to the erythemal action spectrum rather than the synthesis efficacy curve, the synthesis attenuation produced by a given SPF value may differ from the erythemal attenuation. The synthesis-specific SPF attenuation model may take as inputs the SPF rating of the applied sunscreen, the broad-spectrum classification, the chemical versus mineral formulation, the decay state of the sunscreen as calculated by the SPF decay model at each polling interval, and/or the body surface area and areas of application. It may produce as output a value attributed to effective synthesis-available UV-B radiation reaching each protected skin area. The UV-B radiation value may be used as input to the synthesis rate calculation for those areas.

In some embodiments, the second calculation pathway 1710 may be configured to incorporate an activity-adaptive synthesis surface area profile for each activity profile in the activity database described with reference to FIG. 23. The synthesis surface area profile may be configured to encode the estimated percentage of total body surface area that is typically exposed to direct UV-B during that activity, based on the postural geometry and typical clothing patterns characteristic of the activity. The raw synthesis rate calculated from the sensor reading, synthesis efficacy curve, and photosensitivity modifier may be multiplied by the activity-specific exposed surface area fraction to produce a whole-body synthesis rate estimate. In some embodiments, the system 1700 is configured to detect prone and supine position indicators from accelerometer data and to apply a posture correction to the synthesis surface area calculation, adjusting per-area synthesis contributions accordingly rather than applying a fixed whole-body fraction.

In some embodiments, the second calculation pathway 1710 is configured to apply synthesis-specific angular correction factors derived from the angular correction system described with reference to FIG. 24. For synthesis purposes, the correction may be particularly important because the synthesis-effective UV-B fraction may be concentrated at shorter wavelengths that are more directional than the broader UV-B band measured for erythemal purposes. Angular deviation from normal solar incidence may therefore affect synthesis-effective UV-B to a somewhat different degree than erythemal UV-B, and in some embodiments the angular correction model may be configured to apply synthesis-specific correction factors distinct from the erythemal correction factors at the same deviation angle.

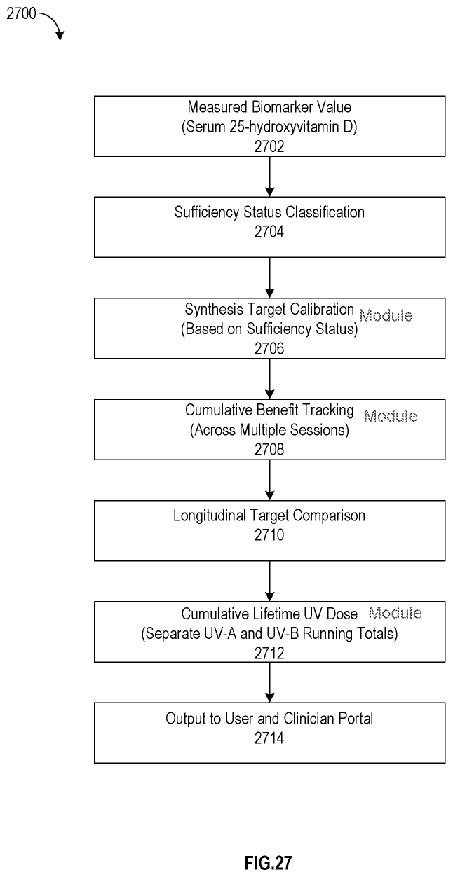

In some embodiments, a multi-objective evaluator module 1716 is configured to evaluate a multi-objective exposure condition by comparing the burn-prevention output 1712 and the Vitamin D synthesis output 1714 against multiple exposure-related thresholds. The exposure-related thresholds may include a first threshold associated with a risk condition corresponding to erythemal skin damage and a second threshold associated with a benefit condition corresponding to a target Vitamin D synthesis level. In some embodiments, the second threshold is calibrated based on a serum 25-hydroxyvitamin D level input, as described with reference to FIG. 27.

In some embodiments, when multiple modifying inputs are simultaneously active for the same body area, the system 1700 is configured to apply them simultaneously rather than selecting a single governing input. Inputs that modify effective UV dose and inputs that modify the threshold value may operate in combination, thereby producing a net remaining exposure time that may reflect all active modifiers concurrently. Where inputs produce conflicting threshold values for the same area, the system 1700 may be configured to resolve the conflict by applying the most conservative result or the input combination that produces the lowest effective exposure ceiling for that area. In certain embodiments, the sole exception to the conservative resolution rule is clinician-driven threshold modification for therapeutic dosing purposes, where the clinician retains authority to govern which value applies.

In some embodiments, the per-area threshold or its effective equivalent may be recalculated dynamically during an active session as input conditions change. Dynamic recalculation may be triggered by events including SPF decay progressing to a new effective SPF value at a predetermined polling interval, user confirmation of sunscreen reapplication resetting the decay clock, user reporting a skin reaction during a session, accelerometer-detected activity change triggering a posture and exposure geometry update, environmental condition change detected via weather API or onboard sensor, for example, user update to clothing coverage, concern area status, or other relevant input, and/or any other input change that may modify the effective threshold or dose calculation for an active body area. In some embodiments, in simplified use cases or low-power operating modes, for example, the threshold is set at session initiation and may be held static for the duration of the session. The choice between static and dynamic threshold behavior may be user-configurable or system-determined based on available inputs and device capability.

In some embodiments, a conflict detector module 1718 may be configured to detect, in response to at least one of the plurality of exposure-related thresholds being inconsistent with at least one other of the plurality of exposure-related thresholds, a conflict condition. A conflict condition may be identified when the UV exposure required to meet the Vitamin D synthesis target would exceed the burn-prevention threshold. In some embodiments, a conflict condition is identified when protective measures such as sunscreen application reduce synthesis-effective UV-B to a level at which the synthesis rate is negligible while burn protection remains adequate. In some embodiments, a conflict condition is identified when a clinician-set UV avoidance prescription reduces the available exposure window to a level insufficient for synthesis.

For example, identified conflict conditions may be classified into severity tiers that govern the type and urgency of resolution outputs generated. A mild conflict, in which the user can achieve partial synthesis but not full session target synthesis within burn-safe parameters, may generate an informational notification. A moderate conflict, in which the SPF-blocked synthesis condition is present, may generate a notification advising the user that their current sunscreen is preventing synthesis and offering specific behavioral options. A severe conflict, in which synthesis is impossible within the current session's burn-safe parameters, may generate a notification advising the user that outdoor synthesis is not available in this session and suggesting alternative approaches.

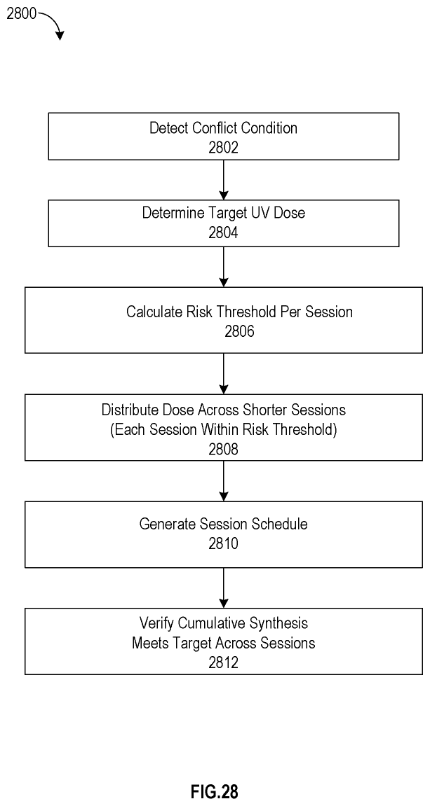

In some embodiments, an optimization logic module 1720 is informed by the dynamic user model 1704 (shown via dashed connection), to utilize optimization logic to resolve the conflict condition. In some embodiments, resolving the conflict condition includes generating a fractionated exposure recommendation distributing a target UV dose across a plurality of shorter sessions, each session remaining within the risk threshold, such that cumulative synthesis across the plurality of shorter sessions meets the synthesis target, as described in greater detail with reference to FIG. 28.

In some embodiments, the optimization logic module 1720 is configured to generate targeted unprotected exposure guidance specifying a protocol for brief, body-area-specific unprotected UV-B exposure designed to maximize synthesis contribution while minimizing burn risk. For example, the system 1700 may be configured to rank body areas by synthesis-to-burn-risk ratio, and/or the IU of synthesis achievable per unit of erythemal risk consumed, and may identify the optimal body area or areas for targeted unprotected exposure. The guidance may specify the recommended body area, the recommended duration of unprotected exposure at the current UV index, the estimated synthesis contribution from that exposure, the burn risk consumed as a percentage of the erythemal threshold for that area, the instructions for sunscreen application to that area following the unprotected window, and/or may provide any other suitable guidance. Targeted unprotected exposure guidance may not be delivered to users with a clinician-set UV avoidance prescription for any area included in the guidance. In some embodiments, the optimization logic module 1720 is configured to generate supplementation guidance when outdoor synthesis is determined to be structurally insufficient to meet the synthesis target.

In some embodiments, when a clinician-set UV avoidance prescription is active and produces a conflict with the Vitamin D synthesis target, the system 1700 may not generate any resolution output that would recommend UV exposure in excess of the clinician's prescription. The system 1700 may instead generate a notification to the user advising that their current UV avoidance protocol prevents outdoor Vitamin D synthesis and recommending that they discuss Vitamin D sufficiency management, including supplementation, with their prescribing clinician. A corresponding flag may be generated in the clinician portal notifying the clinician that the patient's avoidance prescription is preventing synthesis and flagging the patient's current Vitamin D status and cumulative synthesis deficit for clinical review.

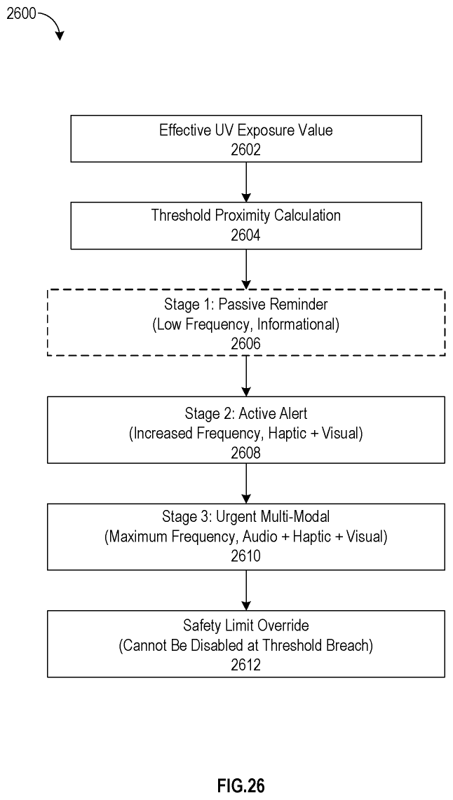

In some embodiments, an output 1722 is generated to influence user behavior based on the resolved conflict condition. The output 1722 may include a predictive alert generated prior to reaching the risk threshold, a Vitamin D synthesis opportunity notification delivered when UV conditions are favorable for synthesis, a fractionated exposure schedule, targeted unprotected exposure guidance, a supplementation guidance notification, and/or the like. In some embodiments, the output 1722 includes a multi-stage escalating notification as described with reference to FIG. 26.

In some embodiments, the output 1722 includes a Vitamin D deficiency risk notification generated by the system's AI and algorithmic analysis layer when the user's cumulative synthesis record indicates an emerging deficiency trajectory. Deficiency risk may be assessed based on one or more of synthesis deficit accumulation, consecutive session underperformance, seasonal synthesis availability decline, post-burn synthesis interruption, and/or the like. Nudges generated in response to deficiency risk signals may be delivered through a behavioral nudge engine using a combination of rules-based triggers and machine learning models trained on patient behavioral data, for example. Nudge content types may include synthesis opportunity alerts delivered when current UV conditions are favorable for synthesis, behavioral adjustment suggestions advising specific behavioral changes that would increase synthesis without increasing burn risk, supplementation reminders generated when the system determines that outdoor synthesis is structurally insufficient to close the deficit, seasonal preparation guidance delivered at the onset of low-synthesis seasons, and/or any other suitable content types known to those in the art.

In some embodiments, the user may manually adjust their effective dose threshold upward beyond the system-calculated recommendation through the companion app. The system 1700 may present a warning that the override is not recommended and/or that the system-calculated threshold reflects the user's current photosensitivity profile and cumulative UV history. The user may choose to proceed with the override despite the warning. All user-initiated overrides may be logged with a timestamp, the magnitude of the override, and the user's current adjusted continuous score at the time of the override. This log may be visible to any clinician with whom the user has an established clinical relationship within the platform. Upon reviewing an override, the clinician may have the ability to restore the system-recommended threshold from the clinician portal, thereby overriding the user's self-adjustment.

Referring now to FIG. 18, while also referring to FIG. 17, a block diagram of the wearable UV sensor assembly 102 is illustrated. The wearable UV sensor assembly 102 may be configured for dual-band UV radiation detection and/or orientation sensing. In some embodiments, the wearable UV sensor assembly 102 provides the radiation-related data used by the system 1700 to determine the effective UV exposure value 1706.

In some embodiments, the wearable UV sensor assembly 102 includes a UV-A sensor element 1802 and a UV-B sensor element 1804 as separate, independently measuring sensor elements. The UV-A sensor element 1802 may be configured to detect UV radiation within the UV-A wavelength band or approximately 315 to 400 nanometers, for example. The UV-B sensor element 1804 may be configured to detect UV radiation within the UV-B wavelength band or approximately 280 to 315 nanometers, for example. Each sensor element 1802, 1804 may produce an independent measurement at each polling interval, thereby enabling the system 1700 to independently measure and separately weight UV-A and UV-B radiation in a threshold determination, dose calculation, notification logic, and/or reporting content. In some embodiments, a single broadband sensor element with software-based spectral differentiation produces separate UV-A and UV-B values from a single physical component. The hardware architecture is claimed functionally to preserve scope across both embodiments and any combination thereof. In some embodiments, the assembly includes more than two sensor elements, enabling measurement across additional wavelength ranges including narrowband UV-B or at approximately 311 nanometers, for example, for phototherapy tracking, visible light bands for red and blue light therapy monitoring, UV-C for occupational monitoring, and/or the like.

In some embodiments, the dual-band sensor architecture provides a precision advantage over weather API data. The wearable UV sensor assembly 102 may be configured to measure UV-A and UV-B levels at the precise location of the UV sensor assembly 102 on the user's body and may account for local environmental factors including surface reflectivity, shading, device angle relative to solar incidence, and/or proximity to artificial UV sources, for example. Weather API UV data may represent an ambient UV index for a geographic location and does not account for these local factors.

In some embodiments, a microcontroller 1806 is configured to receive data from both the UV-A sensor element 1802 and the UV-B sensor element 1804. The microcontroller 1806 may be configured to coordinate data acquisition from all sensor elements 1802, 1804 and to manage communication with external processors. In some embodiments, all UV sensing, decay model execution, session timing, notification state determination, and output element driving functions may be performed by the microcontroller 1806 without requiring data transmission to or processing by an external device.

In some embodiments, the wearable UV sensor assembly 102 further includes an accelerometer 1808 in communication with to the microcontroller 1806. The accelerometer 1808 may be configured to provide three-axis measurement of UV sensor assembly 102 orientation and motion, from which the device's tilt, pitch, and roll relative to a gravity reference frame are derived. The accelerometer 1808 may also enable activity type detection based on movement patterns characteristic of specific activities, as described in greater detail with reference to FIG. 23. In some embodiments, the accelerometer 1808 detects movement patterns characteristic of specific activities and maps those patterns to an activity classification using a proprietary database of activities and their correlated accelerometer signatures.

In some embodiments, the wearable UV sensor assembly 102 further includes a magnetometer 1810 in communication with the microcontroller 1806. The magnetometer 1810 may be configured to provide three-axis measurement of magnetic field direction, from which compass heading may be derived. Together, the accelerometer 1808 and magnetometer 1810 may provide a complete three-dimensional orientation description of the device in space, thereby enabling angular correction of UV sensor assembly 102 readings based on the device's orientation relative to solar incidence, as described in greater detail with reference to FIG. 24. In some embodiments, the accelerometer 1808 and magnetometer 1810 are implemented as two physically separate components. In other embodiments, they are implemented as a single combined inertial measurement unit configured to provide both magnetometer 1810 and accelerometer 1808 functionality in a single chip.

In some embodiments, a GPS module 1812 is provided which is in communication with the microcontroller 1806 for location determination. The GPS module 1812 may be configured to provide geographic coordinates and/or timestamp data used in calculating sun position for angular deviation determination. In some embodiments, the GPS module 1812 is also configured to provide altitude data for reflectivity enhancement detection and atmospheric correction. In some embodiments, the GPS module 1812 is configured to provide location data used to calculate a seasonal synthesis availability factor representing the fraction of days and hours during which ambient UV-B at the user's location contains sufficient synthesis-effective wavelengths to support measurable cutaneous Vitamin D production.

In some embodiments, a wireless transceiver 1814 is configured to communicate data to one or more external processors, such as a companion smartphone or other suitable computing device. The wireless transceiver 1814 may implement one or more wireless communication protocols including Bluetooth, Bluetooth Low Energy, and/or other suitable protocols. In occupational fleet deployment contexts, the wireless transceiver 1814 may be configured to communicate with site hubs, mesh network nodes, and/or central management systems. In some embodiments, communication between the wearable UV sensor assembly 102 and site aggregation infrastructure is multi-directional, enabling supervisor-initiated alerts to be delivered to individual worker devices without requiring worker action.

In some embodiments, the housing lid over the UV sensor aperture of the wearable UV sensor assembly 102 or other suitable component of the wearable UV sensor assembly 102 incorporates a mechanical diffuser element (not shown). The mechanical diffuser element may be, for example, a physical optical component that scatters incoming UV radiation before it reaches the sensor surface. In this manner, the mechanical diffuser element may reduce the angular sensitivity of the UV sensor and enable it to accept UV radiation from a wider range of incident angles rather than responding only to direct normal incidence. This may expand the effective acceptance cone of the UV sensor, thereby improving measurement accuracy at moderate angular deviations without requiring correction model intervention. In some embodiments, the mechanical diffuser element is integrated directly into an aperture window of the housing lid. In these and other embodiments, the dimensions of the mechanical diffuser element and/or aperture window may be enlarged relative to the UV sensor element to increase the solid angle of UV radiation received by the UV sensor element.

Referring now to FIG. 19, while also referring to FIGS. 17 and 18, a dual-output calculation architecture 1900 for simultaneous burn prevention and Vitamin D synthesis calculation is illustrated. The dual-output calculation architecture 1900 may correspond to the first calculation pathway 1708 and second calculation pathway 1710 described above with reference to FIG. 17.

In some embodiments, an effective UV exposure value 1902 serves as the input to two parallel calculation pathways that operate simultaneously from the same underlying sensor measurement obtained from the wearable UV sensor assembly 102. The effective UV exposure value 1902 may correspond to the effective UV exposure value 1706 described with reference to FIG. 17.

In some embodiments, the first calculation pathway processes the effective UV exposure value 1902 through an erythemal action spectrum 1904. The erythemal action spectrum 1904 may be configured to weight UV radiation according to its relative contribution to acute erythemal skin damage, with UV-B radiation weighted more heavily than UV-A radiation. The first calculation pathway may thus produce a burn-prevention output 1908 representing or approximating the remaining safe UV exposure time before erythemal threshold breach. The burn-prevention output 1908 may correspond to the burn-prevention output 1712 described with reference to FIG. 17.

In some embodiments, a second calculation pathway processes the effective UV exposure value 1902 to determine a biologically effective UV dose 1906 based on spectral components. The biologically effective UV dose 1906 may represent the portion of UV-B radiation that is effective for cutaneous Vitamin D synthesis. In some embodiments, the biologically effective UV dose 1906 is calculated based on the UV-B sensor reading from the UV-B sensor element 1804 of the wearable UV sensor assembly 102.

In some embodiments, the second calculation pathway is configured to apply a synthesis efficacy curve 1910 to the biologically effective UV dose 1906. The synthesis efficacy curve 1910 may be distinct from the erythemal action spectrum 1904 and/or may encode the relative effectiveness of each UV-B wavelength in driving Vitamin D synthesis. In some embodiments, the synthesis efficacy curve 1910 reflects peak effectiveness at approximately 295 to 300 nanometers and declining efficacy at wavelengths above 310 nanometers. The synthesis efficacy curve 1910 may include a different shape and/or peak than the erythemal action spectrum 1904.

At any given UV-B dose, the synthesis-effective component and the erythemal-effective component may represent distinct fractions of the total UV-B exposure. The dual-output calculation architecture 1900 may be configured to utilize separate curves for each stream applied to the same raw sensor data, thereby producing independent outputs that may diverge significantly depending on the spectral composition of ambient UV-B at the time of measurement. This divergence may be particularly pronounced in environments where UV-B spectral composition differs from standard solar spectrum ratios, such as at high altitude, in reflective environments, or under artificial UV-B sources including phototherapy lamps, for example.

In some embodiments, the second calculation pathway combines the output of the synthesis efficacy curve 1910 with an exposed body surface area 1914 to produce a Vitamin D synthesis output 1912. The exposed body surface area 1914 may represent the fraction of total body surface area receiving direct UV-B at the time of measurement. In some embodiments, the exposed body surface area 1914 is derived from at least one of an activity profile and a body diagram interface input. In some embodiments, the body diagram interface displays front, back, and side views of a human body outline, and the user indicates areas covered by clothing, areas where sunscreen has been applied and at what SPF, areas of personal concern, and/or the like.

In some embodiments, the user inputs body area data through a body diagram interface in the companion app. The user interface may be configured to display front, back, and side views of a human body outline, for example. In these and other embodiments, the user may indicate areas covered by clothing, areas where sunscreen has been applied and at what SPF, and/or areas of personal concern such as prior skin damage, surgical scars, or high-risk zones. Areas marked as clothed in the body diagram interface may be excluded from the synthesis surface area calculation. Areas marked as sunscreen-protected may be included in the synthesis surface area at the attenuated synthesis rate produced by the synthesis-specific SPF attenuation model. Areas marked as both clothed and sunscreen-protected may be treated as clothed for synthesis purposes since clothing may be treated as complete UV-B blockage in some embodiments.

In some embodiments, the Vitamin D synthesis output 1912 may accumulate at each polling interval as a function of all active modifying inputs. The Vitamin D synthesis output 1912 may correspond to the Vitamin D synthesis output 1714 described with reference to FIG. 17.

In some embodiments, the dual-output calculation architecture 1900 enables the system 1700 to present both the burn-prevention output 1908 and the Vitamin D synthesis output 1912 to the user as parallel real-time indicators. In some embodiments, each output 1908, 1912 may be recalculated at each polling interval and/or updated simultaneously in a companion app interface. In some embodiments, the user may view either output independently or both simultaneously. In some embodiments, notification logic for each output may be independently configurable within a companion application (for example, a mobile application used by the workers). The synthesis output may not generate threshold-breach alerts using the same escalating modalities as the burn-prevention output unless the user configures Vitamin D target-reached notifications, which may be delivered as a positive achievement notification rather than a safety alert. The two output streams may operate from the same sensor data but through distinct calculation pathways. Updates to any shared input, such as a change in angular correction, SPF decay, activity profile, and/or photosensitivity score, may propagate simultaneously to both streams, thereby ensuring that both outputs always reflect current conditions.

Referring now to FIG. 20, while also referring to FIGS. 17 and 19, a dynamic user model 2000 for representing a user's photosensitivity characteristics and enabling dynamic updates over time is illustrated. The dynamic user model 2000 may substantially correspond to the dynamic user model 1704 described above with reference to FIG. 17.

In some embodiments, the dynamic user model 2000 derives a continuous fractional photosensitivity score 2010 from multiple weighted inputs. The continuous fractional photosensitivity score 2010 may be computed as a continuous fractional value rather than a discrete integer skin type category. The continuous fractional photosensitivity score 2010 may be used as a direct numerical input to dose calculation algorithms without rounding to a whole integer before being applied to threshold formulas. This may allow the method 2000 to differentiate meaningfully between users whose questionnaire responses would both round to the same integer skin type but whose continuous scores differ, producing different threshold outputs for each. The continuous fractional photosensitivity score 2010 may be computed internally for dose calculation purposes and may not be user-facing. The user-facing display may present a rounded whole-integer skin type on a 1-6 scale, for example, for clarity and familiarity, while the underlying dose engine may operate on an unrounded continuous value.

In some embodiments, the weighted inputs include a skin reaction to sun exposure 2002. The skin reaction to sun exposure 2002 may represent the user's reported response after sun exposure, ranging from high sensitivity responses such as painful redness and blistering to low sensitivity responses such as rarely or never burning. In some embodiments, the weighted inputs include a freckling characteristic 2004 representing the user's freckle and small mole count and distribution.

In some embodiments, the weighted inputs include a natural eye color 2006 and a natural hair color 2008. In some embodiments, the natural eye color 2006 and natural hair color 2008 are captured through a visual color matrix selection interface rather than text description to reduce user interpretation error. In some embodiments, the weighted inputs further include current use of skincare ingredients with known photosensitizing effects and personal and family medical history of conditions that increase UV sensitivity, including prior skin cancer diagnoses. In some embodiments, the weighted inputs further include depth of tan achieved upon sun exposure, the first body areas to show signs of burning, frequency of sunscreen use, and/or any other suitable input.

In some embodiments, each input is assigned a proprietary weight in the scoring algorithm. Weights may reflect the relative clinical importance of each input in predicting photosensitivity and are initially fixed based on established dermatological literature and clinical input, subject to update over time through machine learning as the system accumulates user data. The weighting model may therefore be both rules-based and data-trained, with updates applied without requiring changes to the user-facing questionnaire.

In some embodiments, skincare ingredient use and medical history of UV-sensitizing conditions are not routed exclusively to a photosensitivity modifier system. These inputs may also directly modify the continuous fractional photosensitivity score 2010 itself. A user currently using retinol or other retinoids, for example, may receive a lower continuous fractional photosensitivity score 2010 than they would absent that input, reflecting increased effective photosensitivity at the scoring level rather than only as a downstream threshold modifier. This may produce a more conservative baseline dose calculation from which subsequent modifiers operate. In some embodiments, the clinical rationale may be that chronic use of photosensitizing skincare ingredients constitutes a baseline shift in photosensitivity rather than a situational acute modifier.

In some embodiments, following completion of the questionnaire, the user is prompted to complete a facial scan using the companion app camera. The facial scan may be processed by an AI image analysis module configured to analyze visually confirmable physical characteristics, including skin color of non-exposed areas, eye color, and/or visible skin features such as freckling and existing sun damage. The AI module may be configured to override questionnaire-derived values for inputs that can be visually confirmed from the image. Inputs that cannot be visually confirmed, such as natural hair color where the user may have dyed hair, skincare product use, and medical history, may not be overridden by the image analysis and may retain their questionnaire-derived values. Where the image analysis produces a result that conflicts with the questionnaire-derived score for a visually confirmable input, the dynamic user model 2000 may automatically apply the more conservative of the two values to that input's contribution to the continuous score. In this manner, the image-based analysis may correct for human error and self-reporting bias in questionnaire responses, particularly for inputs such as skin color where users may misjudge their own baseline tone.

In some embodiments, the dynamic user model 2000 is updateable over time based on a plurality of update triggers shown in dashed boxes. A burn event trigger 2012 may initiate a temporary threshold reduction and a permanent floor reduction following a reported burn event. In some embodiments, the temporary acute reduction may reduce the user's UV exposure threshold for a recovery period following the reported burn, thereby reflecting the skin's acutely compromised state immediately after UV damage. The duration of this temporary reduction may be configurable and/or may scale with reported burn severity. In some embodiments, a moderate burn may trigger a temporary reduction period of approximately seven days, for example, while a more severe reported burn may extend this period.

In some embodiments, following the temporary recovery period, the user's threshold does not return to its prior level but recovers partially to a permanent floor that is lower than the pre-burn threshold. This models the biological accumulation of UV-induced photodamage, in which each burn event produces some degree of irreversible reduction in the skin's UV tolerance. The magnitude of the permanent floor reduction may scale with reported burn severity. The permanent floor may be cumulative across multiple burn events over the life of the account, meaning repeated burns produce a progressively lower floor over time.

In some embodiments, a cumulative UV dose trigger 2014 initiates a downward adjustment to the photosensitivity score when cumulative dose crosses defined thresholds, reflecting progressive photodamage accumulation independent of any individual reported reaction. The cumulative UV dose trigger 2014 may interact with the cumulative lifetime UV dose tracking described with reference to FIG. 27.

In some embodiments, an AI image analysis trigger 2016 initiates an automatic adjustment when the AI image analysis module detects visible changes in skin characteristics such as increased freckling, visible tanning, or signs of photodamage by comparing facial scan images captured at different points in time. In some embodiments, the AI module is configured to override questionnaire-derived values for inputs that can be visually confirmed from the image. Inputs that cannot be visually confirmed, such as natural hair color where the user may have dyed hair, skincare product use, and medical history, are not overridden by the image analysis and retain their questionnaire-derived values.

In some embodiments, a seasonal re-entry trigger 2018 prompts the user to review and confirm or update their current photosensitivity score after a defined period of time or at a seasonal interval. The re-entry prompt may present the user with their current adjusted continuous score, expressed as a whole-integer skin type for display purposes, and may ask them to confirm whether it accurately reflects their current skin state or to update it. In some embodiments, the re-entry process does not return to the original onboarding baseline but begins from the current adjusted score, reflecting the cumulative history of all prior adjustments.

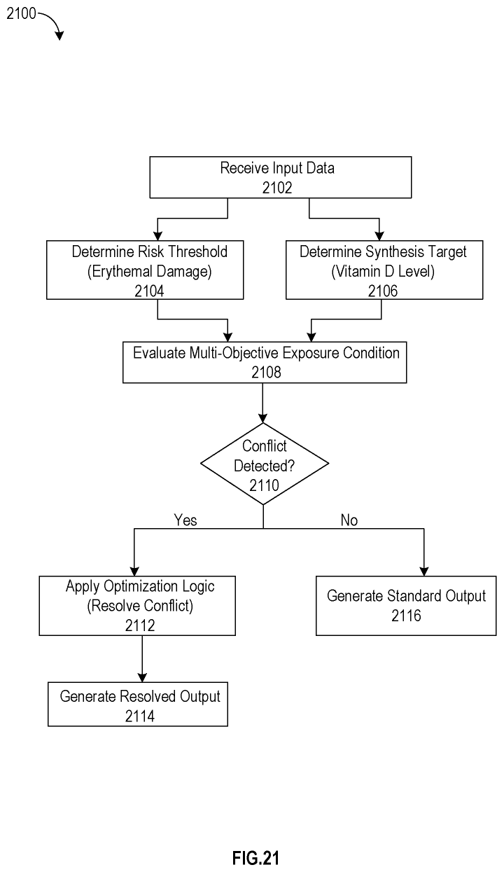

Referring now to FIG. 21, while also referring to FIGS. 17 and 20, a multi-objective threshold determination method 2100 for determining and resolving conflicts between exposure-related thresholds is illustrated. The method 2100 may correspond to the operations performed by the multi-objective evaluator module 1716, conflict detector module 1718, and optimization logic module 1720 described with reference to FIG. 17.

In some embodiments, the method 2100 begins at step 2102 where input data is received. The input data may include at least one of radiation-related data derived from the wearable UV sensor assembly 102, environmental data, user-related data, and/or protection-related data. In some embodiments, the radiation-related data includes independently measured UV-A radiation data and UV-B radiation data obtained from the UV-A sensor element 1802 and UV-B sensor element 1804 described above with reference to FIG. 18.

In some embodiments, at step 2104, a risk threshold associated with erythemal damage is determined. The risk threshold may represent the maximum UV dose the method 2100 estimates a user can tolerate for a given body area without significant risk of erythemal damage. In some embodiments, the risk threshold is derived from the continuous fractional photosensitivity score 2010 described with reference to FIG. 20, modified by all active inputs in a multi-input threshold determination architecture.

In some embodiments, at step 2106, a synthesis target associated with a Vitamin D level is determined. The synthesis target may represent the target Vitamin D synthesis level for the current session. In some embodiments, the synthesis target is calibrated based on a serum 25-hydroxyvitamin D level input, a user age, an exposed body surface area, and the synthesis efficacy curve 1910 described with reference to FIG. 19. In some embodiments, the synthesis target is calibrated based on a sufficiency status classification as described with reference to FIG. 27.

In some embodiments, at step 2108, a multi-objective exposure condition is evaluated by comparing outputs against both the risk threshold and the synthesis target. In some embodiments, the multi-objective exposure condition is evaluated by comparing the burn-prevention output 1908 to the risk threshold and the Vitamin D synthesis output 1912 to the synthesis target.

In some embodiments, at decision step 2110, the method 2100 determines whether a conflict condition is detected. A conflict condition may exist when satisfying one threshold is inconsistent with satisfying another threshold. In some embodiments, a synthesis-impossible conflict is identified when the burn-prevention threshold has been reached or the remaining safe exposure time is insufficient to produce any measurable synthesis contribution at the current UV-B intensity and synthesis rate. In some embodiments, an SPF-blocked synthesis conflict is identified when the effective SPF applied to exposed skin areas is reducing synthesis-effective UV-B to a level at which the synthesis rate is negligible, while the burn-prevention stream indicates remaining exposure capacity. In some embodiments, a partial synthesis conflict is identified when remaining safe exposure time is positive but is shorter than the remaining synthesis time needed to reach the session target.

In some embodiments, if a conflict is detected at step 2110, the method 2100 proceeds to step 2112 where optimization logic is applied to resolve the conflict. The optimization logic may generate a fractionated exposure recommendation as described with reference to FIG. 28, targeted unprotected exposure guidance, and/or supplementation guidance depending on the severity and type of conflict. In some embodiments, conflict conditions are classified into severity tiers that govern the type and urgency of resolution outputs generated.

In some embodiments, at step 2114, a resolved output is generated based on the optimization logic. The resolved output may include a fractionated exposure schedule, targeted unprotected exposure guidance specifying a protocol for brief, body-area-specific unprotected UV-B exposure, and/or supplementation guidance advising the user that oral Vitamin D supplementation reviewed with a clinician is an established alternative pathway to sufficiency.

In some embodiments, if no conflict is detected at step 2110, the method 2100 proceeds to step 2116 where a standard output is generated. The standard output may include remaining safe UV exposure time, remaining time to reach the session Vitamin D target, or both as parallel real-time indicators.

Referring now to FIG. 22, while also referring to FIGS. 17 and 19, a Sun Protection Factor (SPF) decay model 2200 for modeling the time-dependent degradation of protective substances such as sunscreen is illustrated. The SPF decay model 2200 may provide one of the variable modification factors that may be used in determining the effective UV exposure value 1706 described with reference to FIG. 17.

In some embodiments, protective substance data 2202 and a formulation type 2204 are primary inputs to the SPF decay model 2200. The protective substance data 2202 may include SPF rating, broad spectrum classification, active ingredient identification, and/or the like. In some embodiments, the user identifies their sunscreen product by entering a brand name or product identifier, thereby enabling the SPF decay model 2200 to reference known active ingredient profiles for that product. In some embodiments, the user photographs the sunscreen ingredient label using a companion app camera, and the image is transmitted to a cloud-based API or processed on-app by an AI module which identifies active ingredients, their concentrations, and their known stability and/or degradation profiles.

In some embodiments, the formulation type 2204 distinguishes between chemical sunscreens and mineral sunscreens. Mineral sunscreens, such as zinc oxide and titanium dioxide, act as physical barriers, offer immediate protection upon application, and are more stable under UV exposure. Their decay is driven primarily by physical removal through rubbing, perspiration, or water exposure rather than UV-induced chemical breakdown. Chemical sunscreens, such as avobenzone and oxybenzone, absorb UV radiation and convert it to heat, require approximately 20 to 30 minutes to activate after application, and degrade more rapidly under direct UV exposure. Some chemical filter compounds are inherently unstable, with efficacy beginning to decline soon after high-intensity UV exposure.

In some embodiments, multiple degradation variables feed into a time-dependent degradation function 2214. Elapsed time 2206 represents the time since last sunscreen application, tracked from the moment the user reports application or confirms reapplication. Accumulated UV dose 2208 may represent the total UV-A and UV-B dose delivered to the protected area since last sunscreen application, tracked continuously by the UV sensor assembly. Perspiration level 2210 may represent the user's self-assessed sweat rate, which may be input as light, moderate, or heavy, for example. Activity intensity 2212 may represent the physical exertion level inferred from activity type, which may serve as a proxy for sweat rate and/or mechanical friction. In some embodiments, activity intensity 2212 is derived from the activity type detection described with reference to FIG. 23.

In some embodiments, the time-dependent degradation function 2214 is configured to model the decay of sunscreen effectiveness as a function of these variables. Chemical sunscreen decay may be modeled as a function of UV dose accumulation and time, with degradation rate accelerated by high UV intensity, perspiration, and/or water exposure. Mineral sunscreen decay may be modeled primarily as a function of physical removal, driven by perspiration rate, water exposure, and/or mechanical friction inferred from activity type and intensity. Both models may output a remaining effective SPF value rather than a binary protected or unprotected state.

In some embodiments, an effective protection factor 2216 is output for use in exposure calculations. The effective protection factor 2216 represents the remaining effective SPF value at a polling interval, which is applied to modify the incoming UV dose for that interval. As effective SPF decays, the modified UV dose to the area may increase, thereby accelerating consumption of the remaining time-to-burn budget. In some embodiments, the effective protection factor 2216 also affects the synthesis-effective UV-B reaching the skin, thereby reducing the Vitamin D synthesis rate for protected areas.

In some embodiments, the SPF decay model 2200 generates reapplication notifications based on one or more conditions. In some embodiments, a reapplication notification may be generated when the user reaches a user-defined percentage of their calculated reapplication window, with a default threshold of 75% elapsed. In some embodiments, a reapplication notification is generated when accelerometer data indicates a change in activity state consistent with a natural reapplication opportunity, such as the user stopping movement or transitioning from high-intensity to low-intensity activity.

Referring now to FIG. 23, while also referring to FIGS. 17, 18, and 22, an activity-adaptive body mapping system 2300 for adjusting UV exposure thresholds based on detected activity type and inferred body exposure geometry is illustrated. The activity-adaptive body mapping system 2300 may provide one of the variable modification factors used in determining the effective UV exposure value 1706 described with reference to FIG. 17.

In some embodiments, accelerometer data 2302 is received from the accelerometer 1808 integrated within the wearable UV sensor assembly 102 described with reference to FIG. 18. In some embodiments, accelerometer data 2302 is additionally or alternatively received from a smartphone accelerometer via a companion apparatus 100 or other suitable accelerometer.

In some embodiments, an activity type detection module 2304 processes the accelerometer data 2302 to detect movement patterns characteristic of specific activities and maps those patterns to an activity classification. The activity type detection module 2304 may utilize a proprietary database of activities and their correlated accelerometer signatures. In some embodiments, activity input is received through one or more of manual user selection through the companion app, automatic detection via motion data from the on-device accelerometer, and/or automatic detection via motion data from the smartphone accelerometer.

In some embodiments, a body exposure geometry 2306 is determined based on the detected activity type. Each activity profile includes a body exposure geometry model identifying which body areas are most exposed during that activity. For example, a cycling profile identifies the posterior neck, forearms, and scalp as high-exposure areas due to forward-leaning posture. In some embodiments, profiles may be static per activity type or dynamically adjusted based on real-time accelerometer and UV sensor data.

In some embodiments, high-exposure body areas 2308 are identified based on the body exposure geometry 2306. The identification of high-exposure body areas 2308 reflects a proprietary understanding of activity-based high-burn locations and areas most susceptible to UV injury for a given activity. In some embodiments, sensor placement recommendations are generated at session initiation based on the detected or selected activity and its associated high-risk body areas.

In some embodiments, per-area threshold adjustment 2310 applies lower thresholds to high-exposure areas. Body areas identified as high-exposure receive downward threshold adjustments relative to the baseline photosensitivity-derived threshold, producing earlier notifications for those areas. This represents the most conservative approach available, where the most exposed areas of the user's skin govern the time-to-burn calculation for the session rather than an averaged or generalized body reading.

In some embodiments, an earlier notification output 2312 is generated for high-exposure body areas, ensuring that the user receives timely warnings for the areas at greatest risk during the detected activity. In some embodiments, the earlier notification output 2312 includes both remaining safe UV exposure time per body area and sunscreen reapplication timing. In some embodiments, the sunscreen reapplication timing output integrates with the SPF decay model 2200 described with reference to FIG. 22, which incorporates activity type as a proxy for sweat rate and physical exertion in calculating reapplication intervals.

In some embodiments, the activity-adaptive body mapping system 2300 provides expected device orientation profiles for known activity types, which serve as predictive inputs to the angular correction system 2400 described with reference to FIG. 24. These expected profiles are combined with real-time accelerometer and magnetometer data to produce more stable angular deviation estimates during high-motion activity phases.

Referring now to FIG. 24, while also referring to FIGS. 17, 18, and 23, an angular correction system 2400 for producing accurate UV readings from a wearable sensor regardless of device orientation is illustrated. The angular correction system 2400 may provide one of the variable modification factors used in determining the effective UV exposure value 1706 described with reference to FIG. 17.

In some embodiments, UV sensor readings are maximally accurate when the sensor face is oriented perpendicular to the sun's rays, representing zero degrees of angular deviation from direct solar incidence. In practice, users do not always position the device optimally for UV capture. Device orientation varies continuously during activity due to body movement, attachment type, wear position, and user behavior. Without correction, angular deviation may produce systematically low UV readings that underestimate the actual UV dose reaching the user's skin, resulting in artificially extended remaining exposure time estimates and understated burn risk.

In some embodiments, three-dimensional (3D) orientation data 2402 is derived from the accelerometer 1808 and the magnetometer 1810 integrated within or coupled to the wearable UV sensor assembly 102 described above with reference to FIG. 18. The accelerometer 1808 may provide three-axis measurement of device orientation and motion, from which the device's tilt, pitch, and roll relative to a gravity reference frame are derived. The magnetometer 1810 may provide three-axis measurement of magnetic field direction, from which compass heading is derived. Together, the accelerometer 1808 and magnetometer 1810 may provide a complete three-dimensional orientation description of the device in space.

In some embodiments, a calculated sun position 2404 is derived from GPS coordinates and timestamp data obtained from the GPS module 1812 described with reference to FIG. 18. The sun's position in the sky at any moment may be a deterministic function of geographic location, date, and time. Standard solar position algorithms may be applied to these inputs to produce a precise sun position vector in three-dimensional space relative to the user's location. In some embodiments, all sun position calculations are performed on a companion smartphone rather than on the device, with the device's role being exclusively data capture.

In some embodiments, an angular deviation 2406 is determined by combining the 3D orientation data 2402 with the calculated sun position 2404. The angular deviation 2406 may represent the angle between the sensor face normal vector and the direct solar incidence vector, expressed in degrees from zero (representing perfect normal incidence) to 180 degrees (representing the sensor facing directly away from the sun). The angular deviation 2406 may be computed periodically, intermittently, or continuously at each polling interval and may serve as an input to a correction model.

In some embodiments, an activity-aware orientation profile 2408 provides expected device orientation based on detected activity type. The activity profile system described with reference to FIG. 23 may provide expected device orientation profiles for known activity types based on the body area where the device is worn and the postural geometry characteristic of that activity. For example, a cycling activity profile with the device worn on the wrist may anticipate a forward-leaning posture with the wrist at a characteristic angle relative to the torso and therefore relative to the sun. The activity-aware orientation profile 2408 may be used as a predictive input to the correction model, providing a prior estimate of likely angular deviation that is combined with real-time accelerometer and magnetometer data to produce a more stable and accurate angular deviation estimate during high-motion activity phases.

In some embodiments, a corrected UV reading 2410 is produced by applying a correction model to raw UV sensor readings based on the angular deviation 2406 and/or the activity-aware orientation profile 2408. The correction model may be built from device testing data collected across a range of controlled angular configurations and UV conditions that produce a device-specific angular response characterization. The corrected UV reading 2410 may estimate what the sensor would have measured at zero degrees angular deviation from direct solar incidence, given the raw reading at the measured deviation angle. The corrected UV reading 2410 may be used in place of the raw sensor reading for all dose calculations, threshold comparisons, and/or remaining time determinations.

In some embodiments, the correction model maintains reliable correction accuracy for angular deviations up to approximately 90 degrees from direct solar incidence. Within this range, the corrected UV reading 2410 may be used as the primary governing UV data source. For angular deviations beyond approximately 90 degrees, where connectivity is available, the angular correction system 2400 may utilize API-derived UV data as a primary source, supplemented by all available device-derived data to produce a UV estimate for that interval.

In some embodiments, for moderate deviations within the reliable correction range, the system 2400 compensates silently using the correction model without generating a user-facing notification. For deviations approaching or exceeding the reliable correction range, the system 2400 may generate a user-facing notification advising that the device may not be optimally positioned for accurate UV capture and suggesting repositioning.

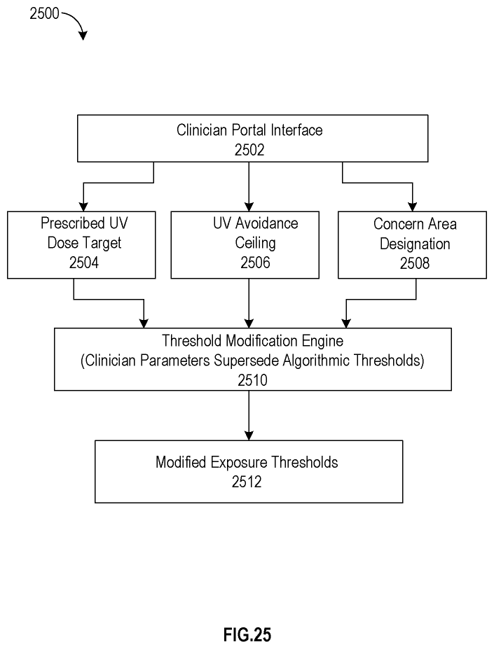

Referring now to FIG. 25, while also referring to FIGS. 17 and 21, a clinician portal system 2500 for enabling clinical oversight and prescriptive dosing capabilities is illustrated. The clinician portal system 2500 may enable clinician-set parameters to be applied as modifying inputs to the plurality of exposure-related thresholds described with reference to FIG. 17.

In some embodiments, a clinician portal interface 2502 is a web- or app-based interface separate from a user-facing companion app. Clinicians may access the clinician portal interface 2502 via credentialed login. The clinician portal interface 2502 may be configured to display all UV exposure data generated by the patient's device and companion app, subject to the patient's paid subscription tier. Data visible to the clinician may include raw UV-A and UV-B dose data per body area, time-of-day exposure patterns, peak exposure hours, peak burn-risk periods, notification response behavior, sunscreen compliance indicators, concern area designations, current threshold settings, and/or aggregated exposure history. In some embodiments, patient data is analyzed using a combination of algorithms and AI-based analysis to generate clinically relevant insights, including identification of peak exposure hours, trend analysis across sessions, sunscreen compliance patterns, and/or behavioral pattern identification.

In some embodiments, clinician alerts are passive rather than active. The clinician portal system 2500 may not push real-time alerts to clinicians for individual threshold breach events. Instead, the system 2500 may send periodic email notifications indicating that patient data is available for review, that a patient has flagged a concern, or that an AI-generated finding has been queued for clinician review. The clinician portal interface 2502 may include a direct messaging interface allowing the clinician to send recommendations, instructions, or follow-up questions to the patient.

In some embodiments, the clinical relationship between a clinician and patient is established in one of two ways. A credentialed clinician onboarded to the platform may enroll a patient directly, or vice versa. Alternatively, patients whose clinician is not onboarded may be assigned a platform-affiliated clinician. Both relationship types may support the same portal functionality.

In some embodiments, through the clinician portal interface 2502, a clinician may set multiple clinician-set parameters for a user. A prescribed UV dose target 2504 may represent a clinician-prescribed daily or weekly UV dose target for a specific patient, specifying the wavelength range, dose quantity, and treatment schedule. In some embodiments, the prescribed UV dose target 2504 is used for therapeutic UV dosing for conditions such as psoriasis, eczema and atopic dermatitis, vitiligo, cutaneous T-cell lymphoma, chronic pruritus, lichen planus, scleroderma, morphea, granuloma annulare, polymorphous light eruption, and/or the like. In some embodiments, the system 2500 is configured to track cumulative dose delivered to the target body area in real time using the UV sensor assembly and notifies the patient when their prescribed dose for a session has been reached.

In some embodiments, a UV avoidance ceiling 2506 represents a hard UV avoidance ceiling for patients requiring strict UV avoidance rather than therapeutic exposure. Conditions requiring strict UV avoidance may include Xeroderma Pigmentosum, extreme photosensitivity disorders, and certain active skin cancers, for example. The UV avoidance ceiling 2506 may supersede the standard photosensitivity-based threshold system and triggers earlier and more urgent alerts than standard notifications. In some embodiments, the system does not override or relax a clinician-set UV avoidance ceiling 2506 based on algorithmic output. Where the system's standard dose recommendation and the clinician's avoidance prescription conflict, the clinician's prescription may govern.

In some embodiments, a concern area designation 2508 represents clinician-defined high-risk zones for a specific patient, such as areas of prior skin damage, surgical scars, or areas with elevated skin cancer risk. Concern areas may receive lower notification thresholds, triggering earlier warnings regardless of SPF application status. In some embodiments, based on high-risk zone designations, the user receives zone-specific sunscreen reapplication reminders.

In some embodiments, the clinician-set parameters feed into a threshold modification engine 2510. The threshold modification engine 2510 may apply clinician parameters as modifying inputs to exposure-related thresholds such that clinician parameters supersede algorithmically determined thresholds for medically significant parameters. In some embodiments, the AI module may autonomously update non-medically-significant parameters including concern area designations, notification thresholds within a predefined safe range, and/or behavioral recommendations. Modifications to prescribed dose targets, hard avoidance ceilings, or clinician-set thresholds may require clinician review and approval through the portal.

In some embodiments, modified exposure thresholds 2512 are output from the threshold modification engine 2510 and applied to the user's exposure management. Where the system's 2500 conservative resolution of other active inputs would produce a lower threshold than the clinician's prescription, the system 2500 may notify the clinician of the conflict and present both values, with the clinician retaining authority to govern which value applies. This conflict resolution interaction may be logged in the clinician portal.

Referring now to FIG. 26, while also referring to FIGS. 17 and 21, an escalating notification architecture 2600 for delivering progressively urgent notifications as a user approaches and exceeds UV exposure thresholds is illustrated. The escalating notification architecture 2600 may provide the notification logic for the output 1722 described with reference to FIG. 17.

In some embodiments, the notification system 2600 is not a single alarm triggered at threshold breach but a multi-stage escalating architecture in which notification frequency, modality, and content increase progressively as the user approaches and then exceeds their UV exposure threshold. The number of escalation stages may not be fixed. Stages may be user-programmable, system-recommended based on established behavioral science principles, and/or dynamically adjusted by the system's machine learning layer based on the individual user's historical notification response behavior.

In some embodiments, an effective UV exposure value 2602 serves as the input to the escalating notification architecture 2600. The effective UV exposure value 2602 may correspond to the effective UV exposure value 1706 described with reference to FIG. 17.

In some embodiments, a threshold proximity calculation 2604 determines the distance between the effective UV exposure value 2602 and the applicable exposure-related thresholds. The threshold proximity calculation 2604 expresses the user's current position as a percentage of threshold consumed. In some embodiments, default stages are defined as percentage-of-threshold milestones at which notifications are generated, such as notifications at 50%, 75%, and 90% of threshold consumed, a threshold breach notification, and post-breach continued monitoring notifications at defined intervals.

In some embodiments, as the effective UV exposure value 2602 approaches thresholds, notification escalates through progressive stages. Stage 1: Passive Reminder 2606 represents an early-stage notification employing low frequency informational notifications. In some embodiments, Stage 1 notifications employ only a companion app push notification. Stage 1 notifications may be delivered when the user reaches a defined percentage of their calculated threshold, such as 50% threshold consumed.

In some embodiments, Stage 2: Active Alert 2608 represents a mid-stage notification with increased frequency and additional modalities. In some embodiments, Stage 2 notifications add haptic feedback on the device combined with visual notifications. Stage 2 notifications may be delivered when the user reaches a higher percentage of threshold consumed, such as 75% threshold consumed.

In some embodiments, Stage 3: Urgent Multi-Modal 2610 represents a late-stage or breach notification employing maximum frequency using audio, haptic, and visual modalities simultaneously. In some embodiments, Stage 3 notifications include rapid continuous LED patterns, maximum brightness, and audible alerts. The specific modality combination assigned to each stage may be user-configurable, with a default escalation pattern recommended by the system based on behavioral effectiveness data.

In some embodiments, a safety limit override 2612 may help to ensure that critical notifications cannot be disabled by the user at threshold breach. The safety limit override 2612 represents a system design constraint that preserves minimum safety requirements and helps to prevent configurations that would result in no notification at threshold breach. In some embodiments, post-breach notifications continue according to the configured post-breach stage structure until the user ends the session or moves to a UV environment where sensor readings indicate negligible ongoing dose accumulation.

In some embodiments, the system's 2600 machine learning layer substantially continuously analyzes the user's historical notification response behavior to optimize notification timing and content. In other embodiments, the machine learning layer analyzes the user's historical notification response behavior periodically, intermittently, or randomly. Signals analyzed may include the time elapsed between notification delivery and user action, the modality combination that produces the fastest response for the individual user, the threshold percentages at which the user most reliably acts, and/or the times of day and activity contexts in which the user is most and least responsive to notifications.

Referring now to FIG. 27, while also referring to FIGS. 17, 19, and 21, a serum-calibrated Vitamin D tracking system 2700 for calibrating synthesis targets based on measured biomarker values and tracking cumulative synthesis accumulation longitudinally is illustrated. The serum-calibrated Vitamin D tracking system 2700 may provide the calibration for the second threshold associated with a benefit condition described with reference to FIG. 17.

In some embodiments, a measured biomarker value 2702 representing serum 25-hydroxyvitamin D is received. Serum 25-hydroxyvitamin D may be the principal circulating form of Vitamin D used to assess sufficiency. In some embodiments, the measured biomarker value 2702 may be input via direct integration with a laboratory data provider that transmits 25-hydroxyvitamin D test results electronically to the system upon test completion, without requiring manual data entry by the user. In some embodiments, the measured biomarker value 2702 is input via user-initiated manual entry of a 25-hydroxyvitamin D value from a test result obtained outside the platform. In some embodiments, the measured biomarker value 2702 is input via clinician entry through the clinician portal interface 2502 described with reference to FIG. 25.

In some embodiments, a sufficiency status classification 2704 classifies the user's Vitamin D status based on the measured biomarker value 2702 into one of a defined set of categories. Categories may include but are not limited to severely deficient, deficient, insufficient, sufficient, and optimal. The classification thresholds may be configurable system parameters based on established clinical reference ranges. The default thresholds may be derived from published endocrinology and dermatology guidelines. In some embodiments where a clinician has established a clinical relationship within the platform, the clinician may configure custom classification thresholds for that patient through the clinician portal.

In some embodiments, a synthesis target calibration module 2706 may be configured to adjust the Vitamin D synthesis target based on the sufficiency status classification 2704. The synthesis target calibration module 2706 may produces a deficit value representing the estimated gap between the user's current serum level and the lower bound of the sufficient range, or between the current level and a clinician-defined target level where one has been set. In some embodiments, the system 2700 converts the deficit value into an estimated cumulative UV-B exposure requirement using an established photobiological model that incorporates the user's continuous fractional photosensitivity score 2010 described with reference to FIG. 20, an age-related synthesis capacity modifier, and/or typical exposed body surface area. The cumulative UV-B exposure requirement may be distributed across a defined target period, such as a week or a month, to produce a session-level synthesis target.

In some embodiments, a cumulative benefit tracking module 2708 is configured to track cumulative Vitamin D synthesis across all sessions over the life of the account. The cumulative benefit tracking module 2708 may be configured to operate at two levels of granularity: session-level synthesis representing the total estimated Vitamin D IU produced in each individual session, stored with session timestamp and environmental context, for example; and cumulative synthesis tracking representing the running total across all sessions, compared against the user's cumulative target derived from their serum baseline and the target period.

In some embodiments, a longitudinal target comparison 2710 compares the cumulative benefit tracked by cumulative benefit tracking 2708 against the longitudinal target derived from the sufficiency status classification 2704. The longitudinal target comparison 2710 may calculate the synthesis surplus or deficit relative to the target for defined periods such as daily, weekly, or monthly intervals. This comparison may drive deficiency risk notification and/or behavioral nudge outputs.

In some embodiments, a cumulative lifetime UV dose module 2712 is configured to maintain separate UV-A and UV-B running totals across sessions. The cumulative lifetime UV dose module 2712 may be tracked separately from synthesis accumulation, reflecting the distinct long-term health consequences of UV-A exposure (primarily photoaging and long-term dermal damage) and UV-B exposure (primarily cumulative skin cancer risk and erythemal damage history). In some embodiments, the cumulative lifetime UV dose module 2712 is tracked at two levels of granularity: per-area lifetime dose tracking cumulative UV-A and UV-B dose delivered to each individually monitored body area, and whole-body aggregate lifetime dose tracking total UV-A and UV-B dose across all monitored areas.

In some embodiments, an output to user and clinician portal 2714 surfaces the results of the serum-calibrated Vitamin D tracking system 2700 to both the user and any clinician with an established clinical relationship. The output may include current session synthesis progress displayed as a progress indicator toward the session target, weekly and monthly synthesis summaries compared against targets, synthesis deficit or surplus expressed in IU and contextualized in terms of estimated time needed to close the deficit, serum status indicators where a serum value has been entered, synthesis trend visualizations showing accumulation across sessions over the account history, and/or any other suitable or desired output.

Referring now to FIG. 28, while also referring to FIGS. 17, 21, and 27, a fractionated exposure method 2800 for resolving conflict conditions between risk thresholds and synthesis targets by distributing UV dose across multiple shorter sessions is illustrated. The fractionated exposure method 2800 may correspond to one resolution approach of the optimization logic module 1720 described with reference to FIG. 17.

In some embodiments, at step 2802, a conflict condition is detected. The conflict condition may exist when the UV exposure required to meet the Vitamin D synthesis target would exceed the burn-prevention threshold in a single session. In some embodiments, the conflict condition is detected by the conflict detector module 1718 described with reference to FIG. 17 or at decision step 2110 described with reference to FIG. 21.

In some embodiments, at step 2804, a target UV dose is determined based on the synthesis target. The target UV dose may represent the total UV-B exposure required to produce the target Vitamin D synthesis contribution. In some embodiments, the target UV dose is derived from the synthesis target calibration 2706 described above with reference to FIG. 27.

In some embodiments, at step 2806, a risk threshold per session is calculated. The risk threshold per session may help to ensure that each individual session remains within the user's burn-safe exposure window, accounting for the user's continuous fractional photosensitivity score 2010 described with reference to FIG. 20 and all active threshold modifiers. In some embodiments, the risk threshold per session is derived from the risk threshold determined at step 2104 described with reference to FIG. 21.

In some embodiments, at step 2808, the target UV dose is distributed across a plurality of shorter sessions. Each shorter session may be configured to remain within the risk threshold per session calculated at step 2806, such that no individual session breaches the erythemal threshold. In some embodiments, the distribution accounts for the user's typical activity patterns, available outdoor time, and/or forecasted UV conditions.

In some embodiments, at step 2810, a session schedule is generated. The session schedule may specify the number of sessions required to meet the synthesis target at the current conflict severity level, the recommended duration of each session at the current or forecasted UV index, the recommended time of day for each session accounting for peak synthesis-effective UV-B availability balanced against burn risk, and/or the recommended body surface area to expose during each session prioritizing areas with the lowest concern area designation and the highest synthesis surface area contribution.

In some embodiments, at step 2812, the method 2800 verifies that cumulative synthesis across the shorter sessions meets the synthesis target. The method 2800 may track the user's adherence to the fractionated exposure schedule across sessions, adjusting the recommendation dynamically as actual synthesis accumulation deviates from the projected schedule. In some embodiments, the cumulative synthesis verification interacts with the cumulative benefit tracking 2708 described with reference to FIG. 27.

In some embodiments, fractionated exposure recommendations are generated by the system's algorithmic analysis layer and are presented to the user as informational guidance within the companion app. In some embodiments, where a clinical relationship exists, fractionated exposure schedules may be reviewed and modified by the clinician through the clinician portal interface 2502 described with reference to FIG. 25.

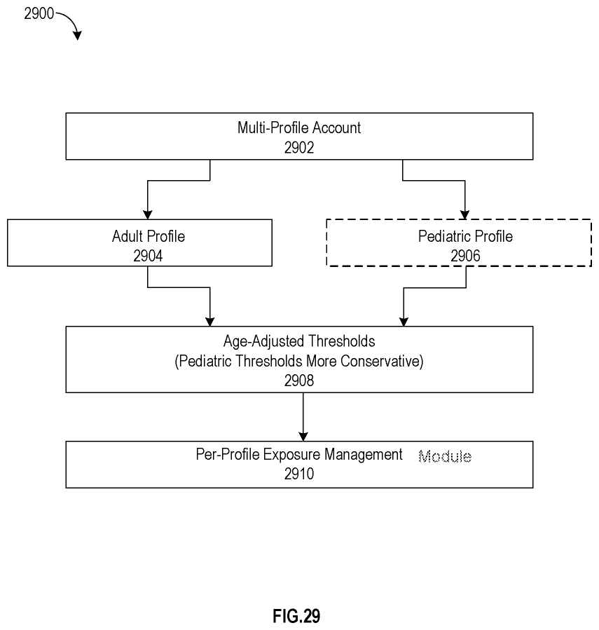

Referring now to FIG. 29, while also referring to FIGS. 17, 20, and 27, a multi-profile management system 2900 for managing a plurality of linked user profiles within a single account, including pediatric profiles with age-adjusted thresholds, is illustrated.

In some embodiments, a multi-profile account 2902 includes multiple linked user profiles. A primary account holder may create and administer the multi-profile account 2902 and may create linked profiles for family members including minor children and other dependents. In some embodiments, family members who are adults may independently create their own linked accounts that connect to the primary account, with the primary account holder able to view linked member data subject to the member's consent and the account's subscription tier.

In some embodiments, an adult profile 2904 represents a standard user profile using standard exposure-related thresholds derived from the user's continuous fractional photosensitivity score 2010 described with reference to FIG. 20 and other modifying inputs. The adult profile 2904 may include its own longitudinal dose history, sunscreen preferences, activity profile, concern area designations, and notification settings. Each linked family member profile may be a fully independent profile with its own threshold calculations, dose tracking, and notification settings.

In some embodiments, a pediatric profile 2906, shown in dashed outline to indicate special handling, represents a profile created for a minor child. The pediatric profile 2906 may be created and managed exclusively by the primary account holder until an age-based transition of profile control occurs. In some embodiments, at a defined age threshold, the method 2900 supports a configurable transition of profile control to the child, allowing the child to assume management of their own profile while maintaining the option for the primary account holder to retain view access with the child's consent.

In some embodiments, age-adjusted thresholds 2908 help to ensure that pediatric thresholds are more conservative than equivalent adult thresholds for the same photosensitivity score. Children's skin has distinct UV sensitivity characteristics relative to adults, reflecting differences in melanin distribution, skin thickness, DNA repair capacity, and lifetime cumulative dose vulnerability. In some embodiments, the age-adjusted thresholds 2908 apply age-specific modifiers to the photosensitivity-derived baseline threshold, thereby producing more conservative thresholds for younger users independent of their photosensitivity score. In some embodiments, age-specific questionnaire inputs are incorporated into the photosensitivity scoring algorithm for pediatric profiles, reflecting the distinct physical characteristics relevant to UV sensitivity assessment in children. In some embodiments, in the clinical context, a clinician sets age-specific threshold parameters through the clinician portal interface 2502 described with reference to FIG. 25 for a pediatric patient based on clinical judgment and the child's specific skin condition and UV risk profile.

In some embodiments, a per-profile exposure management module 2910 applies the appropriate thresholds to each profile independently. In some embodiments, a single UV sensor device may be shared among family members with profile switching at session initiation, where the companion app supports profile switching allowing the current user to select their profile before initiating a session. Session data collected during that session may be attributed to the selected profile and contributes to that profile's longitudinal dose history. In some embodiments, each family member has a dedicated device permanently associated with their individual profile, which is the preferred configuration for accurate lifetime cumulative dose tracking.

In some embodiments, the multi-profile management system 2900 includes a family-level Vitamin D sufficiency dashboard presenting the synthesis status and sufficiency classification for all linked family member profiles simultaneously. The family-level dashboard may display each family member's current sufficiency classification where a serum value has been entered, each member's synthesis progress toward their individual target, an aggregate family synthesis summary, and/or any active deficiency risk flags for any family member.

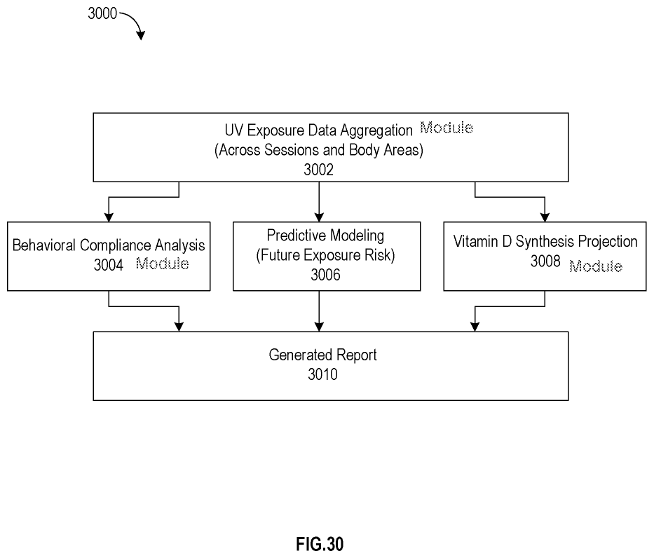

Referring now to FIG. 30, while also referring to FIGS. 17, 21, 27, and 29, a report generation system 3000 for generating customized UV exposure reports that aggregate data collected across sessions, body areas, and time periods is illustrated.

In some embodiments, a UV exposure data aggregation module 3002 is configured to collect exposure data across sessions and body areas. The UV exposure data aggregation module 3002 may include data such as total UV-A dose, total UV-B dose, UV-A to UV-B ratio, total exposure time, peak UV index encountered, peak exposure hour, peak burn-risk period, body area exposure breakdown, and/or a comparison to prior periods. In some embodiments, the UV exposure data aggregation module 3002 incorporates data from the cumulative lifetime UV dose 2712 described with reference to FIG. 27.

In some embodiments, the aggregated data feeds into three parallel analysis components. A behavioral compliance analysis module 3004 may be configured to evaluate user adherence to recommendations. In some embodiments, the behavioral compliance analysis module 3004 is configured to analyze sunscreen application compliance rate, sunscreen reapplication compliance rate, notification response rate, dose override frequency and magnitude, and/or an overall behavioral compliance score derived from a compliance model. In some embodiments, the behavioral compliance analysis module 3004 is configured to track user behavior across sessions and adjust input weighting based on observed adherence to system recommendations, as described with reference to the dynamic user model 1704 in FIG. 17.

In some embodiments, predictive modeling 3006 projects future exposure risk based on the user's historical exposure patterns, behavioral compliance profile, seasonal UV index data for the user's location, and current skin profile status including cumulative damage history. Predictive outputs may include projected cumulative dose at current behavioral trajectory over a defined forward period, estimated time before the user's cumulative dose reaches a clinically significant milestone, predicted peak exposure risk windows for the upcoming period derived from historical session timing and seasonal UV data, and behavioral change impact modeling showing the estimated reduction in projected cumulative dose achievable by specific recommended behavioral changes.

In some embodiments, a Vitamin D synthesis projection module 3008 is configured to estimate synthesis accumulation at the current behavioral trajectory. The Vitamin D synthesis projection module 3008 may include projected synthesis accumulation over a defined forward period expressed both in estimated IU and as a projected serum level change, estimated time to reach a target serum 25-hydroxyvitamin D level at current synthesis rates presented with an uncertainty range, seasonal synthesis availability forecast identifying periods of reduced synthesis opportunity before they occur, and/or behavioral impact modeling showing estimated synthesis improvement achievable by specific behavioral changes. In some embodiments, the Vitamin D synthesis projection module 3008 is configured to incorporate data from the cumulative benefit tracking 2708 and longitudinal target comparison 2710 described with reference to FIG. 27.

In some embodiments, all three analysis components contribute to a generated report 3010. The generated report 3010 may be delivered in one or more formats including in-app display within the companion app with data visualizations and trend charts, patient portal web display in a browser-accessible format with expanded visualization and download options, PDF download for sharing with clinicians not on the platform, email delivery providing a summary notification with a secure link to the full report in the portal, structured data export in a format compatible with electronic health record import where EHR integration is available, and/or management dashboard display presenting occupational reports in a supervisor-facing interface.

In some embodiments, report generation is triggered by one or more conditions including session completion, periodic time-based triggers at defined intervals such as weekly or monthly, user request, clinician request through the clinician portal interface 2502 described with reference to FIG. 25, threshold breach events, and/or AI behavioral pattern detection identifying patterns warranting proactive surfacing. In some embodiments, report content and/or depth are governed by the user's subscription tier and the deployment context, for example.

In some embodiments, predictive content is clearly labeled as modeled projections based on current behavioral patterns and is not presented as clinical predictions or medical diagnoses. The system communicates the assumptions underlying predictive outputs and the degree to which projected outcomes depend on behavioral change.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A system for dynamically managing ultraviolet (UV) radiation exposure of a user, comprising:
a wearable UV sensor assembly configured to detect UV radiation data associated with a user;
one or more processors;
non-transitory computer-readable storage media storing code, the code being executable by the one or more processors to perform operations comprising:
receiving input data associated with the user, the input data comprising at least one of radiation-related data derived from the wearable UV sensor assembly, environmental data, user-related data, and protection-related data;
maintaining a dynamic user model associated with the user, wherein the dynamic user model is updateable over time based on the input data, and wherein the dynamic user model represents a current response of the user to UV exposure;
determining, based on the dynamic user model and the input data, a plurality of exposure-related thresholds, the plurality of exposure-related thresholds comprising a first threshold associated with a risk condition and a second threshold associated with a benefit condition;
determining an effective UV exposure value based on the input data and at least one variable modification factor;
evaluating a multi-objective exposure condition by comparing the effective UV exposure value to the plurality of exposure-related thresholds;
detecting, in response to at least one of the plurality of exposure-related thresholds being inconsistent with at least one other of the plurality of exposure-related thresholds, a conflict condition;
utilizing an optimization logic based on the dynamic user model to resolve the conflict condition; and
generating an output configured to influence user behavior based on the resolved conflict condition.

2. The system of claim 1, wherein the radiation-related data includes at least one of ultraviolet A (UV-A) radiation data and ultraviolet B (UV-B) radiation data independently obtained from separate sensor elements, and wherein the at least one variable modification factor includes a user-configurable concern weighting configured to adjust a relative contribution of the UV-A radiation data and the UV-B radiation data to the effective UV exposure value.

3. The system of claim 1, wherein the environmental data includes at least one of geographic location, time of day, altitude, atmospheric conditions, and weather data.

4. The system of claim 1, wherein the protection-related data comprises information associated with a protective substance applied to the user, wherein the information comprises a formulation type of the protective substance and a time-dependent degradation of the protective substance modeled as a function of at least one of an elapsed time, an accumulated UV dose, a perspiration level, and an activity intensity.

5. The system of claim 1, wherein the dynamic user model includes a representation of at least one of a skin type, a pigmentation level, and a physiological response to UV exposure.

6. The system of claim 1, wherein the dynamic user model is continuously updated based on historical exposure data associated with the user.

7. The system of claim 1, wherein the first threshold corresponds to a risk of skin damage and the second threshold corresponds to a target associated with Vitamin D synthesis.

8. The system of claim 1, wherein the output includes at least one of a notification, a recommendation, a behavioral instruction, and a control signal.

9. A computer-implemented method for dynamically managing ultraviolet (UV) radiation exposure of a user, comprising:
receiving input data associated with a user, the input data comprising at least one of radiation-related data derived from a wearable UV sensor assembly, environmental data, user-related data, and protection-related data;
maintaining a dynamic user model associated with the user, wherein the dynamic user model is automatically updateable over time based on the input data, and wherein the dynamic user model represents a current response of the user to UV exposure;
determining, based on the dynamic user model and the input data, a plurality of exposure-related thresholds, the plurality of exposure-related thresholds comprising a first threshold associated with a risk condition and a second threshold associated with a benefit condition;
determining an effective UV exposure value based on the input data and at least one variable modification factor;
evaluating a multi-objective exposure condition by comparing the effective UV exposure value to the plurality of exposure-related thresholds;
detecting, in response to at least one of the plurality of exposure-related thresholds being inconsistent with at least one other of the plurality of exposure-related thresholds, a conflict condition;
utilizing an optimization logic based on the dynamic user model to resolve the conflict condition; and
generating an output configured to influence user behavior based on the resolved conflict condition.

10. The computer-implemented method of claim 9, wherein determining the effective UV exposure value includes applying an orientation-based adjustment on the radiation-related data, the orientation-based adjustment derived from a three-dimensional (3D) orientation of the wearable UV sensor assembly and a calculated sun position, to generate a corrected UV reading used in determining the effective UV exposure value, and wherein the orientation-based adjustment is activity-aware based on an expected device orientation profile associated with a detected activity type.

11. The computer-implemented method of claim 9, further comprising:
estimating a biologically effective UV dose based on a spectral component of the effective UV exposure value; and
estimating a Vitamin D synthesis level based on the biologically effective UV dose, a synthesis efficacy curve, and an exposed body surface area, wherein the exposed body surface area is derived from at least one of an activity profile and a body diagram interface input.

12. The computer-implemented method of claim 11, further comprising:
generating a report aggregating UV exposure data across sessions and body areas, the report including at least one of behavioral compliance analysis, predictive modeling of future exposure risk, and a projected Vitamin D synthesis accumulation at a current behavioral trajectory.

13. The computer-implemented method of claim 9, wherein resolving the conflict condition includes prioritizing one of the plurality of exposure-related thresholds based on user-specific parameters, and generating a fractionated exposure recommendation distributing a target UV dose across a plurality of shorter sessions, each session remaining within the risk threshold, such that cumulative synthesis across the plurality of shorter sessions meets a synthesis target.

14. The computer-implemented method of claim 9, wherein the output includes a staged notification based on proximity to one or more thresholds.

15. A program product comprising a non-transitory computer-readable storage medium and program code, the program code being executable by one or more processors to perform operations comprising:
receiving input data associated with a user, the input data comprising at least one of radiation-related data derived from a wearable UV sensor assembly, environmental data, user-related data, and protection-related data;
maintaining a dynamic user model associated with the user, wherein the dynamic user model is updateable over time based on the input data, and wherein the dynamic user model represents a current response of the user to UV exposure;
determining, based on the dynamic user model and the input data, a plurality of exposure-related thresholds, the plurality of exposure-related thresholds comprising a first threshold associated with a risk condition and a second threshold associated with a benefit condition;
determining an effective UV exposure value based on the input data and at least one variable modification factor;
evaluating a multi-objective exposure condition by comparing the effective UV exposure value to the plurality of exposure-related thresholds;
detecting, in response to at least one of the plurality of exposure-related thresholds being inconsistent with at least one other of the plurality of exposure-related thresholds, a conflict condition;
utilizing an optimization logic based on the dynamic user model to resolve the conflict condition; and
generating an output configured to influence user behavior based on the resolved conflict condition.

16. The program product of claim 15, wherein the operations further comprise integrating external radiation data from a remote data source.

17. The program product of claim 15, wherein the dynamic user model is updated using a machine learning process.

18. The program product of claim 15, wherein the effective UV exposure value comprises a time-weighted cumulative exposure.

19. The program product of claim 15, wherein the output comprises a predictive alert generated prior to reaching the risk threshold.

20. The program product of claim 15, wherein the operations further comprise generating a user interface comprising a visualization of exposure and threshold conditions.
